(19) United States
(12) Patent Application Publication
(10) Pub. No.: US 2026/0241107 A1
(43) Pub. Date: Aug. 20, 2026

(54) CARTRIDGE FOR A DISPENSING DEVICE WITH MOVABLE GRIP PLATE

(71) Applicant: medmix Switzerland AG, Haag (CH)

(72) Inventors: Emanuel BASTOS, Buchs (CH); Ralf INAUEN, Zürich (CH); Josef MOSER, Künten (CH)

(21) Appl. No.: 19/145,125

(22) PCT Filed: Nov. 16, 2023
(86) PCT No.: PCT/EP2023/082093

§ 371 Date: Jul. 1, 2025

(30) Foreign Application Priority Data
Dec. 20, 2022 (GB) 2219267.8

(51) Int. Cl. A61M 5/31; A61M 5/19

(52) U.S. Cl. CPC A61M 5/3137; A61M 5/19; A61M 2005/3139; A61M 2205/586

(57) ABSTRACT

A cartridge for a dispensing device includes a grip plate, the grip plate is at least axially moveably mounted at the cartridge and axially fixable to the cartridge.

### CROSS-REFERENCE TO RELATED APPLICATIONS

This application is a U.S. National Stage application of International Application No. PCT/EP2023/0082093 filed Nov. 16, 2023, which claims priority to GB Application No. 2219267.8, filed Dec. 20, 2022.

### TECHNICAL FIELD

The present disclosure relates to a cartridge for a dispensing device and to a method of adapting a distance between an end of the cartridge and an actual position of a grip plate.

### BACKGROUND

Dispensing devices exist in different forms and sizes. Furthermore, dispensing devices can be used in many different applications, like medical applications, dental applications or industrial applications.

However, an operation of a dispensing device can almost always be the same independently of the application. The dispensing device can typically comprise a container or a cartridge containing material. The material can be extracted out of the container through an opening arranged at one end of the container. To extract the material through the opening, the material can be pressed out of the container using, for example, a plunger with a piston.

The dispensing device can also comprise a grip plate arranged at an end of the container where the piston is inserted into the container. The grip plate can help to control a force applied to the plunger. For example, a user can press with his thumb on the plunger and simultaneously hold the grip plate with his index and middle fingers to control the force applied by pressing the plunger.

### SUMMARY

It has been determined that the control of the force can be arduous leading to less than desirable dispensing results.

Accordingly, there is a need for methods and devices to improve an ergonomic handling of a dispensing device and to leverage the force required to dispense the material out of the dispensing device.

The present disclosure provides a cartridge, a method and a dispensing device according to the independent claims. Embodiments are given in the subclaims, the description and the drawings.

In one aspect, the present disclosure is directed at a cartridge for a dispensing device. The cartridge comprises a grip plate, wherein the grip plate is at least axially moveably mounted at the cartridge and axially fixable to the cartridge.

The cartridge can be configured to receive material, wherein the material can be liquid or viscous. The cartridge can comprise a first end and a second end. Further, the cartridge can comprise a first opening at the first end and a second opening at the second end. The cartridge can be configured to receive a piston through the first opening at the first end. The cartridge can be configured that a needle or a nozzle can be attached to the second end such that the material contained in the cartridge can be dispensed out of the cartridge through the second opening at the second end and through the needle or nozzle. Any appropriate material for a specific application of the cartridge can be received and contained by the cartridge. The cartridge can comprise one or more containers, wherein each of the containers can be configured to receive and contain material.

The dispensing device can be a syringe, for example, used by a dentist for narcotic injections or to fill a tooth. The grip plate can be ergonomically formed. Further, the grip plate can be arranged on an outer surface of the cartridge.

An axial direction can be defined as a direction directing from the first end of the cartridge to the second end of the cartridge. In other words, the axial direction can be described by a longitudinal axis of the cartridge.

As the grip plate is axially moveably mounted at the cartridge and axially fixable to the cartridge an optimal ergonomic positioning of the grip plate on the surface of the cartridge can be achievable. For example, the grip plate can be fixed to the cartridge at a position that can be optimal regarding a hand size of a user or optimal regarding a required force, required to extract the material out of the cartridge. Thus, a handling of a dispensing device comprising the cartridge can be improved.

According to an embodiment, a cross section of the cartridge perpendicular to an axial direction can be round, oval, or D-shaped. The cross section of the cartridge can be described by a section perpendicular to the longitudinal axis of the cartridge. The D-shaped cross section can comprise a round-shaped section at one side of the cross section and a rectangular shaped section at the other side of the cross section. Alternatively, the cross-section of the cartridge can be oval or elliptical.

According to an embodiment, the cartridge can comprise two containers arranged in parallel, the two containers can have equal or unequal volumes and/or equal or unequal outer cross-sectional areas. Each of the two containers can be configured to receive and contain material, wherein one container can contain a different material than the other container. Each of the two containers can be cylinders, i.e. can comprise a cylindrical shape. However, the two containers can also be of different shape.

According to an embodiment, an outer shape of the cartridge can be round or oval. The cartridge can enclose at least one container inside the cartridge. The outer shape of the cartridge can be different from an inner shape of the cartridge. The inner shape of the cartridge can be adapted to a shape of the at least one container inside the cartridge. For example, if the at least one container is D-shaped, the inner shape of the cartridge can be D-shaped. If the cartridge comprises two coaxially arranged containers, the inner shape of the cartridge can be formed as a horizontal eight that can encompass the two coaxially arranged containers. The outer shape of the cartridge can also be elliptical shaped. A round-shaped, oval-shaped or elliptical-shaped outer shape of the cartridge can beneficial regarding the handling of the dispensing device.

According to an embodiment, the grip plate can be unlockable to release a fixed distance between one end of the cartridge and the grip plate. The grip plate can further be fixable at a position between the first end of the cartridge and the second end of the cartridge to fix another distance between one end of the cartridge and the grip plate. In this way the grip plate can be positioned at an optimal position between the first end of the cartridge and the second end of the cartridge.

According to an embodiment, the grip plate can comprise at least two oppositely arranged handles. The grip plate can comprise two, three, four or more handles. The at least two oppositely arranged handles can extrude in a direction perpendicular to the longitudinal axis of the cartridge. The at least two handles of the grip plate can be arranged at the cartridge such that a user can grasp the at least two handles easily with his fingers. Such handles can allow a simple use of the cartridge or of the dispensing device comprising the cartridge.

According to an embodiment, the at least two oppositely arranged handles can be square-shaped or round-shaped. Such a shape can be beneficial when using the cartridge.

According to an embodiment, the grip plate can be circumferentially closed and placed over the cartridge. Such a grip plate can be beneficial when moving the grip plate on the cartridge since the grip plate may not be detached from the cartridge when the grip plate is moving.

According to an embodiment, the grip plate can be movable back and/or forth along an axial direction of the cartridge. The axial direction of the cartridge can be a longitudinal axis of the cartridge. The expression “forth” can mean that the grip plate is movable in a direction to the second end of the cartridge along the longitudinal axis of the cartridge. The expression “back” can mean that the grip plate is movable in a direction to the first end of the cartridge along the longitudinal axis of the cartridge. The feature of moving the grip plate back and forth can be beneficial when adjusting the optimal distance between one end of the cartridge and the grip plate or between a plunger and the grip plate. Further, moving back the grip plate can be beneficial if the cartridge is refillable. For example, the cartridge can be refilled with material once the cartridge has been emptied and the grip plate can be repositioned by moving the grip plate back along the axial direction of the cartridge. The grip plate can be movable back to a position arranged at the first end of the cartridge.

According to an embodiment, the grip plate can be fixable to the cartridge using a lip. The lip can enclose the outer surface of the cartridge. The lip can be formed from a flexible material, for example, a synthetic material. The lip can be attached to an inner surface of the grip plate. Such a lip can be beneficial to move the grip plate continuously along the axial direction of the cartridge.

According to this embodiment, the lip can be mounted on the grip plate and the lip can be arranged between the grip plate and the cartridge. The lip can be arranged such that the lip can progressively expand as more force can be applied to the grip plate to move the grip plate. The force applied to the grip plate can be a counterforce to the force applied to plunger when extracting the material out of the cartridge. By means of such a feature, the grip plate can be moved along the axial direction of the cartridge when a relatively small force is applied. On the other hand, the grip plate can be fixed in a position on the cartridge when a relatively high force is applied to the grip plate.

According to an embodiment, the lip can be sloped on at least one side. The sloped lip can be configured to build a tension in an axial direction. The tension can prevent the lip and thus the grip plate from moving in this axial direction. Therefore, the grip plate can be fixed in this axial direction. On the other hand, the slope can be configured to enable a movement of the grip plate in the other axial direction.

According to an embodiment, the grip plate can be continuously moveable between a first end of the cartridge and a second end of the cartridge and the grip plate can be fixable to the cartridge at any position between the first end of the cartridge and the second end of the cartridge by clamping the lip between the grip plate and the cartridge. Clamping can mean that there can be a force or a tension between the lip and the outer surface of the cartridge and between the lip and the grip plate, wherein the force or the tension prevents a movement of the grip plate. Such an embodiment can be beneficially used to adjust a distance between the grip plate and the first end of the cartridge frequently.

According to an embodiment, the cartridge can further comprise a plurality of grooves arranged in parallel to one other perpendicular to a longitudinal axis on an outer surface of the cartridge. Such grooves can be beneficially used to define positions on the cartridge where the grip plate can be fixed.

According to this embodiment, each groove of the plurality of grooves can be configured to receive the lip. The lip can be received from the respective groove when the grip plate is moved along the longitudinal axis of the cartridge over the respective groove. The grip plate can be fixed at the position of the respective groove when the lip is received by the respective groove. The grip plate can be unlockable from this position if a force higher than a retention force is applied to the grip plate to press the lip out of the groove. The retention force can be a force that prevents the lip from moving out of the groove.

According to an embodiment, each groove of the plurality of grooves can be completely or partly circumferentially arranged at the cartridge. A completely circumferentially arranged groove at the cartridge can cause a higher retention force. Thus, fixing the grip plate to the cartridge can be more stable. On the other hand, a partly circumferentially arranged groove at the cartridge can be beneficial when the grip plate is partially circumferentially arranged at the outer surface of the cartridge.

According to an embodiment, the grip plate can be moveable between a first end of the cartridge and a second end of the cartridge along a longitudinal axis of the cartridge in predefined steps defined by the plurality of grooves and the grip plate can be fixable to the cartridge at any predefined step between the first end of the cartridge and the second end of the cartridge by receiving the lip through one groove of the plurality of grooves. The plurality of grooves and thus the predefined steps can be arranged equidistantly along the outer surface of the cartridge. A distance between each two grooves of the plurality of grooves can be different. For example, the distance between two grooves at the first end of the cartridge can be smaller than the distance of two grooves in the middle of the cartridge, i.e. in the middle between the first end of the cartridge and the second end of the cartridge.

According to an embodiment, the grip plate can be fixable to the cartridge using a locking rail. The locking rail can extend in an axial direction, particularly along the longitudinal axis of the cartridge, from a vicinity of the first end of the cartridge almost to the second end of the cartridge. Such a locking rail can be beneficially used to move the grip plate continuously along the cartridge.

According to this embodiment, the locking rail can comprise a longitudinal recess at an outer surface of the cartridge and the grip plate comprises a nose, wherein the nose can be embedded in the locking rail. The longitudinal recess can be configured to receive the nose of the grip plate. The nose of the grip plate can be controlled by the longitudinal recess such that the grip plate is moveable in the longitudinal direction of the recess back and/or forth along the axial direction of the cartridge. The nose can be configured to prevent a movement of the grip plate perpendicular to the longitudinal axis of the cartridge. The grip plate can be fixed at any position of the locking rail using a clamp or a latch. The clamp or the latch can be configured to secure the nose of the grip plate at a selected position on the locking rail.

According to an embodiment, the grip plate can be fixable to the cartridge using a fixing device. Such a fixing device can comprise a tongue and a groove or a toothing and cams, arranged at the cartridge and the grip plate respectively. One part of the fixing device can be arranged at the grip plate and a counterpart of the fixing device can be arranged at the cartridge. The part and the counterpart can engage with each other such that the grip plate can be fixable to the cartridge.

According to this embodiment, the fixing device can comprise a toothing arranged at the cartridge and one or more cams arranged at the grip plate, wherein the toothing and the one or more cams can mesh with one another if the grip plate is fixed. The one or more cams can be moved over the toothing, wherein the one or more cams can be receivable by a notch between two teeth of the toothing. Such a toothing and cams arrangement of the fixing device can be comparable to a cable-tie principle. Therefore, the tooting and cams arrangement can allow a movement of the grip plate in one direction, for example, in a direction from the first end of the cartridge to the second end of the cartridge. A movement of the grip plate from the second end of the cartridge to the first end of the cartridge can not be possible, i.e. the grip plate can be fixed against a movement in that direction.

According to an embodiment, the fixing device can comprise a latch to fix the grip plate to the cartridge and unfix the grip plate from the cartridge. The latch can be configured to press the toothing against the cams when the grip plate is fixed to the cartridge. The latch can be applied at any position where the toothing and the cams engage. The latch can comprise an open position and a closed position. The toothing and the cams can engage in the closed position of the latch. If the latch is in the open position, the toothing and the cams can be moveable in relation to each other.

According to an embodiment, the grip plate can be fixable to the cartridge using at least one bulge or at least one indentation. The at least one bulge or that at least one indentation can be arranged on an outer surface of the cartridge. The grip plate can be fixable to the cartridge by snapping onto the at least one bulge or by snapping into the at least one indentation. The grip plate can comprise a respective recess or a respective protrusion. The respective recess can be configured to receive a bulge of the at least one bulges when the grip plate is moved over the bulge. Accordingly, the at least one indentation of the cartridge can be configured to receive the protrusion of the grip plate when the grip plate is moved over the indentation. Such a configuration of at least one bulge or at least one indentation on the surface of the cartridge can be beneficial to predefine positions on the cartridge to which the grip plate can be fixed.

According to this embodiment, the at least one bulge or the at least one indentation can be arranged completely or partly circumferentially at the cartridge. A completely circumferentially arranged bulge or indentation can enable a stable fixing of the grip plate to the cartridge. On the other hand, a partly circumferentially arranged bulge or indentation at the cartridge can be beneficial when the grip plate is partially circumferentially arranged at the outer surface of the cartridge.

According to an embodiment, the grip plate can be deformable. Such a deformable grip plate can be beneficial to move the grip plate along the cartridge and to fix the grip plate to the cartridge. In particular, a clamp or a latch can not be required to fix the grip plate to the cartridge.

According to this embodiment, the grip plate can comprise a V-shaped gap between two oppositely arranged handles. The V-shaped gap can be beneficial to deform the grip plate. The V-shaped gap can be widened when the grip plate is deformed, for example, when the grip plate is in a deformed state.

According to an embodiment, the grip plate can be movable back and/or forth along an axial direction of the cartridge when the grip plate is in a deformed state and the grip plate can be fixable to the cartridge when the grip plate is in an undeformed state. The grip plate can be brought into the deformed state by pushing the two oppositely arranged handles of the grip plate in a direction according to the longitudinal axis of the cartridge, in particular by pushing the grip plate in a direction from the first end of the cartridge to the second end of the cartridge, Thus, the V-shaped gap can be widened and the grip plate can be movable along the axial direction of the cartridge. Otherwise, if the grip plate can not be pushed in that direction, but can be pulled in an opposite direction, for example in the direction from the second end of the cartridge to the first end of the cartridge, or if the grip plate can not be pulled or pushed at all, the grip plate can be in the undeformed state. The grip plate can be fixed to the cartridge if the grip plate is in the undeformed state.

According to an embodiment, the cartridge can comprise a plurality of cavities arranged in parallel to one another perpendicular to a longitudinal axis on an outer surface of the cartridge and the grip plate can comprise at least one hook, wherein the at least one hook can engage at least one of the plurality of cavities when the grip plate is undeformed to fix the grip plate to the cartridge. The plurality of cavities can be formed by a toothing on the outer surface of the cartridge. The at least one hook can be arranged at an inner side of the grip plate, wherein the inner side of the grip plate can be in contact to the outer side of the cartridge. The at least one hook can extend from the grip plate, in particular from the inner side of the grip plate, in a direction perpendicular to the longitudinal axis of the cartridge. The at least one hook can be release from one cavity of the plurality of cavities by moving the grip plate in a direction along the longitudinal axis of the cartridge from the first end of the cartridge to the second end of the cartridge. Thus, the at least one hook can jump to an adjacent cavity of the one cavity and the at least one hook can engage in the adjacent cavity to fix the grip plate to the cartridge at a position of the adjacent cavity.

According to another embodiment, the grip plate can comprise at least one deformable cantilever. The at least one deformable cantilever can be deformed when the grip plate is moving along an outer surface of the cartridge. The at least one cantilever can be used to fix the grip plate to the cartridge.

According to this embodiment, the at least one deformable cantilever can be fixed to the grip plate at one end of the at least one deformable cantilever and the at least one deformable cantilever can comprise a hook at another end of the at least one deformable cantilever. The at least one hook can be arranged at an inner side of the cantilever, wherein the inner side of the cantilever can face the outer side of the cartridge. The at least one hook can extend from the cantilever, in particular from the inner side of the cantilever, in a direction perpendicular to the longitudinal axis of the cartridge.

According to an embodiment, the cartridge can comprise a plurality of notches arranged in parallel to one another perpendicular to a longitudinal axis on an outer surface of the cartridge. The plurality of notches can be formed by a toothing on the outer surface of the cartridge. Each notch of the plurality of notches can be configured to receive the at least one hook.

According to an embodiment, the at least one deformable cantilever can be movable in a first direction along an axial direction of the cartridge, wherein the hook of the at least one deformable cantilever can be configured to slide over at least one of the plurality of notches. The first direction can be a direction along the longitudinal axis of the cartridge from the first end of the cartridge to the second end of the cartridge. The at least one deformable cantilever can move in the first direction along the axial direction of the cartridge when the grip plate moves in the first direction. The hook can be released from one notch of the plurality of notches by moving the grip plate, and therefore also the at least one cantilever, in the first direction. Thus, the at least one hook can jump to an adjacent notch of the one notch and the at least one hook can engage in the adjacent notch to fix the grip plate to the cartridge at a position of the adjacent notch.

According to an embodiment, the at least one deformable cantilever can be fixable in a second direction along an axial direction of the cartridge, wherein the hook of the at least one deformable cantilever can be configured to engage at least one of the plurality of notches. The second direction can be a direction along the longitudinal axis of the cartridge from the second end of the cartridge to the first end of the cartridge

According to an embodiment, the grip plate can comprise at least one U-shaped channel. The U-shaped channel can be arranged such that two arms forming the U-shape can be arranged perpendicular to the longitudinal axis of the cartridge. Such a U-shaped channel can be beneficial to move the grip plate on the cartridge and to fix the grip plate to the cartridge.

According to an embodiment, the cartridge can comprise at least one pin arranged on an outer surface of the cartridge. The at least one pin can define a position on the cartridge where the grip plate can be fixed to the cartridge. The cartridge can comprise a first pin arranged on the outer surface of the cartridge at the first end of the cartridge. The cartridge can comprise a second pin arranged on the outer surface of the cartridge at the second end of the cartridge.

According to an embodiment, the at least one U-shaped channel can be configured to receive the at least one pin. Such a U-shaped channel arranged on the grip plate can be beneficial in order to hold the grip plate on the cartridge.

According to an embodiment, the grip plate can be rotatable about a longitudinal axis of the cartridge, wherein a rotation of the grip plate can be controlled by the at least one pin in the at least one channel. The grip plate can be rotatable such that the at least one pin can slide along an inner surface of the U-shaped channel. The U-shaped channel can comprise a first tube and a second tube. The first tube can be arranged at the arm of the U-shaped channel directing towards the first end of the cartridge. The second tube can be arranged at the arm of the U-shaped channel directing towards the second end of the cartridge. The tubes can be configured to receive the pin, in particular, the pin can slide through the tubes. Thus, such tubes can be beneficial to move the grip plate along the longitudinal axis of the cartridge by sliding the pin through the second tube, through the U-shaped channel and through the first tube, or vice versa.

According to an embodiment, the cartridge can comprise a first pair of oppositely arranged pins and a second pair of oppositely arranged pins, wherein the first pair of oppositely arranged pins can be located at a first end of the cartridge and the second pair of oppositely arranged pins can be located at a second end of the cartridge, wherein the grip plate can be moveable from the first pair of oppositely arranged pins to the second pair of oppositely arranged pins and the grip plate can be fixable using the second pair of oppositely arranged pins. The pins of each pair of pins can be arranged perpendicular to the longitudinal axis of the cartridge on the outer surface of the cartridge. Using a pair of pins instead of a single pin can result in a more stable fixation of the grip plate to the cartridge.

According to an embodiment, the grip plate can be rotatably arranged between a first rotational position and a second rotational position on an outer surface of the cartridge. The grip plate can be rotatable about the longitudinal axis of the cartridge.

According to an embodiment, the grip plate can be moveable along a direction to the longitudinal axis of the cartridge between a first end of the cartridge and a second end of the cartridge when the grip plate is in the first rotational position, and the grip plate can be fixable at a position between the first end of the cartridge and the second end of the cartridge when the grip plate is in the second rotational position. Such a grip plate can be easy to use to lock and unlock the grip plate on the cartridge.

According to an embodiment, the grip plate can comprise a first plurality of division bars arranged in parallel to one other on an inner surface of the grip plate along a longitudinal axis of the cartridge. The first plurality of division bars can be arranged on the inner surface or on an inner side of the grip plate, wherein the inner surface or the inner side of the grip plate can face an outer side or an outer surface of the cartridge. The first plurality of division bars can be arranged in sections on the inner surface of the grip plate, for example, the division bars of the first plurality of division bars can not be circumferentially closed on the inner surface of the grip plate. Each division bar of the first plurality of division bars can extend from the grip plate, in particular from the inner surface or the inner side of the grip plate in a direction towards the outer surface of the cartridge and perpendicular to the longitudinal axis of the cartridge. A distance between two division bars of the first plurality of division bars can define a predefined step. The grip plate can be moveable along the cartridge and fixable to the cartridge in this predefined steps.

According to an embodiment, the cartridge can comprise a second plurality of division bars arranged in parallel to one another on the outer surface of the cartridge along the longitudinal axis of the cartridge. The second plurality of division bars can be arranged in sections on the outer surface of the cartridge, for example, the division bars of the second plurality of division bars can not be circumferentially closed at the outer surface of the cartridge. Each division bar of the second plurality of division bars can extend from the cartridge, in particular from the outer surface of the cartridge in a direction towards the inner surface of the grip plate and perpendicular to the longitudinal axis of the cartridge. A distance between two division bars of the second plurality of division bars can define a predefined step. The distance between two division bars of the second plurality of division bars can be equal to the distance of two division bars of the first plurality of division bars. The grip plate can be moveable along the cartridge and fixable to the cartridge in this predefined steps.

According to an embodiment, the first plurality of division bars can engage a plurality of indentations formed between the second plurality of divisional bars when the grip plate is in the second rotational position. Each indentation of the plurality of indentations can be formed between two division bars of the second plurality of division bars. The grip plate can be fixed at the cartridge when at least a portion of the first plurality of division bars engages at least one indentation of the second plurality of division bars. The grip plate can be fixed to the cartridge when the grip plate is in the second rotational position.

According to an embodiment, the grip plate can comprise an internal thread. The internal thread can be arranged on an inner surface of the grip plate, wherein the inner surface or the inner side of the grip plate can face the outer surface of the cartridge. The grip plate can be rotatable about the longitudinal axis of the cartridge and moveable in a direction along the longitudinal axis of the cartridge using the internal thread. Such a grip plate can be beneficial in that the grip plate can be continuously moveable along the cartridge.

According to an embodiment, the cartridge can comprise an external threat. The external thread can be arranged on an outer surface of the cartridge, wherein the outer surface of the cartridge can face the inner surface of the grip plate. The grip plate can be rotatable about the longitudinal axis of the cartridge and moveable in a direction along the longitudinal axis of the cartridge using the internal thread and the external thread. Such a grip plate can be beneficial in that the grip plate can be continuously moveable along the cartridge.

According to an embodiment, the grip plate can be axially moveable by screwing the internal thread onto the external thread. An axial movement of the grip plate can depend on a threat pitch of the internal thread and the external threat.

According to an embodiment, the grip plate can be configured to snap to a ridge arranged on an outer surface of the cartridge. The cartridge can comprise at least one ridge arranged on the outer surface of the cartridge. The at least one ridge can be arranged completely or partly circumferentially on the outer surface of the cartridge. The at least one ridge can be arranged in a plane perpendicular to the longitudinal axis of the cartridge. The grip plate can comprise an opening configured to receive the cartridge. The opening of the grip plate can be smaller than a diameter of the cartridge, in particular a smallest distance of the opening can be smaller than the diameter of the cartridge. The grip plate can be configured to snap to the at least one ridge in a direction perpendicular to the longitudinal axis of the cartridge by pushing the grip plate onto the ridge. The opening of the grip plate can widen if the grip plate is pushed onto the ridge. The opening can narrow if the ridge is received by the grip plate. Such a grip plate can be beneficial in that the grip plate is removeable from the cartridge and the grip plate can be used with a different cartridge.

According to an embodiment, the grip plate can enclose the cartridge by more than 50% of a peripheral surface of the cartridge when the grip plate is snapped onto the ridge on the outer surface of the cartridge. The grip plate can be in a mounted position when the grip plate is snapped onto the at least one ridge. The grip plate can be in an unmounted position when the grip plate is removed from the at least one ridge. The grip plate can enclose the cartridge by more than 50% of the peripheral surface of the cartridge when the grip plate is in the mounted position.

According to an embodiment, the cartridge can comprise at least two ridges arranged completely or partly circumferentially on the outer surface of the cartridge. The at least two ridges can define positions at the cartridge where the grip plate can be fixed. Thus, the at least to ridges can define predetermined steps on the outer surface of the cartridge where the grip plate can be fixed.

According to an embodiment, the grip plate can comprise at least one chamfer configured to receive at least one of the at least two ridges. The at least one chamfer can be arranged on an inner surface of the grip plate, wherein the inner surface or the inner side of the grip plate can face the outer surface of the cartridge. The at least one chamfer can be configured to fix or to secure the grip plate to the cartridge when the at least one chamfer receives the at least one of the at least two ridges. The at least one chamfer can encompass the at least one of the at least two ridges at least in sections when the at least one chamfer receives one of the at least two ridges.

According to an embodiment, a first ridge of the at least two ridges can be located at a first end of the cartridge and a second ridge of the at least two ridges can be located at a second end of the cartridge. Such an embodiment can be beneficial to optimize a handling for a dispensing device for different filling levels of the cartridge of the dispensing device. For example, if the cartridge is fully filled with material, the first ridge arranged at the first end of the cartridge can be used to fix the grip plate to the cartridge. If the filling level of the cartridge is decreasing, the grip plate can be moved to the second ridge and the second ridge can be used to fix the grip plate to the cartridge.

According to an embodiment, the grip plate can be fixable at the first end of the cartridge or at the second end of the cartridge by snapping the grip plate onto the first ridge or onto the second ridge such that the at least one chamfer can receive the first ridge or the second ridge.

According to an embodiment, the grip plate can be moveable between the first end of the cartridge and the second end of the cartridge by removing the grip plate from the first ridge and snapping the grip plate onto the second ridge.

According to an embodiment, the grip plate can comprise a first chamfer and a second chamfer configured to receive a corresponding first ridge of the at least two ridges and a second ridge of the at least two ridges. Such a grip plate can be beneficial to stabilize a fixation of the grip plate to the cartridge. The grip plate can be fixed using the first ridge and the second ridge.

According to an embodiment, the grip plate can be fixable at the first end of the cartridge by snapping the first chamfer of the grip plate onto the first ridge and snapping the second chamfer of the grip plate onto the second ridge such that the first chamfer can receive the first ridge and the second chamfer can receive the second ridge.

According to an embodiment, the grip plate can be moveable between the first end of the cartridge and the second end of the cartridge by removing the first chamfer of the grip plate from the first ridge and the second chamfer of the grip plate from the second ridge, and by turning the grip plate such that the first chamfer of the grip plate can receive the second ridge and the second chamfer of the grip plate can receive the first ridge.

According to an embodiment, the grip plate can be fixable at the second end of the cartridge by snapping the first chamfer of the grip plate onto the second ridge and by snapping the second chamfer of the grip plate onto the first ridge such that the first chamfer can receive the second ridge and the second chamfer can receive the first ridge.

According to an embodiment, the grip plate can comprise a first chamfer and a second chamfer configured to receive a ridge, wherein the ridge can be arranged at a second end of the cartridge, the first chamfer can be arranged at a first end of the grip plate and the second chamfer can be arranged at a second end of the grip plate. The first chamfer and the second chamfer can be arranged in parallel to one other on an inner surface of the grip plate. Such a grip plate can be beneficial to fix the grip plate to the cartridge in two different position using one ridge only.

According to this embodiment, the grip plate can be fixable at the first end of the cartridge by snapping the second chamfer of the grip plate onto the ridge such that the second chamfer receives the ridge. In such an embodiment, the first end of the chamfer can correspond to the first end of the cartridge and the second end of the grip plate can correspond to the second end of the cartridge.

According to an embodiment, the grip plate can be moveable between the first end of the cartridge and the second end of the cartridge by removing the second chamfer of the grip plate from the ridge, and turning the grip plate such that the first chamfer of the grip plate can receive the ridge.

According to an embodiment, the grip plate can be fixable at the second end of the cartridge by snapping the first chamfer of the grip plate onto the ridge such that the first chamfer can receive the ridge. In such an embodiment, the first end of the chamfer can correspond to the second end of the cartridge and the second end of the grip plate can correspond to the first end of the cartridge.

According to an embodiment, the grip plate can be arranged at one end of an open tube.

According to this embodiment, the open tube can be configured to snap to an outer surface of the cartridge.

According to an embodiment, the open tube can enclose the cartridge by more than 50% of a peripheral surface of the cartridge when the open tube is snapped onto the outer surface of the cartridge.

According to an embodiment, the cartridge can comprise a rib arranged completely or partly circumferentially at a first end on the outer surface of the cartridge.

According to an embodiment, the grip plate can be fixable at a first position at the first end of the cartridge by snapping the open tube onto the outer surface at the first end of the cartridge such that the one end of the open tube contacts the rib.

According to an embodiment, the grip plate can be fixable at a second position at the first end of the cartridge by snapping the open tube onto the outer surface at the first end of the cartridge such that another end of the open tube oppositely arranged to the one end of the open tube contacts the rib.

According to an embodiment, the grip plate can be moveable in predefined steps and fixable at the predefined steps. Such a grip plate can be advantageous in that the fixation of the grip plate can be reliably repeated. For example, the grip plate can be fixable always at the same predefined steps.

According to another embodiment, the grip plate can be continuously moveable on the cartridge and fixable at any position on the cartridge. Such a grip plate can be advantageous in that the fixation of the grip plate is not limited to predefined steps. Thus, the grip plate can be fixable at any position between the first end of the cartridge and the second end of the cartridge.

In another aspect, the present disclosure is directed at a method of adapting a distance between an end of a cartridge and an actual position of a grip plate, in particular a cartridge in accordance with at least one of the preceding embodiments. The method comprises the following step: moving the grip plate at least axially along the cartridge, in particular wherein the grip plate is axially fixable to the cartridge. The distance between the end of the cartridge and the actual position of the grip plate can be adapted to a hand size of a user of a dispensing device that can comprise the cartridge. Thus, the grip plate can be adapted for each user individually.

According to an embodiment, the method can further comprise the following step: fixing the grip plate to fix the distance between the end of the cartridge and the axial position of the grip plate.

According to an embodiment, the method can further comprise the following steps: moving the grip plate from a first position to a second position; and fixing the grip plate at the second position, wherein the first position can be arranged at a first end of the cartridge and the second position can be arranged at a second end of the cartridge or the second position can be arranged between the first end of the cartridge and the second end of the cartridge.

According to an embodiment, the fixing of the grip plate can be carried out using a lip.

According to this embodiment, the fixing of the grip plate can be carried out using a groove configured to receive the lip, wherein the groove can be arranged on an outer surface of the cartridge.

According to an embodiment, the fixing of the grip plate can be carried out using a locking rail.

According to an embodiment, the fixing of the grip plate can be carried out using a fixing device.

According to an embodiment, the fixing of the grip plate can be carried out using a toothing arranged at the cartridge and one or more cams arranged at the grip plate, wherein the toothing and the one or more cams can mesh with one other if the grip plate is fixed to the cartridge.

According to an embodiment, the fixing of the grip plate can be carried out using a latch.

According to this embodiment, the method can further comprise the following steps: opening the latch to unfix the grip plate from the cartridge before moving the grip plate at least axially along the cartridge; and closing the latch to fix the grip plate to the cartridge after moving the grip plate at least axially along the cartridge.

According to an embodiment, the fixing of the grip plate can be carried out using at least one bulge or at least one indentation.

According to an embodiment, the fixing of the grip plate can be carried out using a deformable grip plate in an undeformed state.

According to an embodiment, the fixing of the grip plate can be carried out using at least one deformable cantilever.

According to an embodiment, the fixing of the grip plate can be carried out using at least one U-shaped channel.

According to an embodiment, the fixing of the grip plate can be carried out using a plurality of division bars.

According to an embodiment, the fixing of the grip plate can be carried out using an internal threat and an external threat.

According to an embodiment, the fixing of the grip plate can be carried out using at least one ridge.

According to an embodiment, the fixing of the grip plate can be carried out using an open tube and a rib.

In another aspect, the present disclosure is directed at a dispensing device. The dispensing device comprises a cartridge according to one of the embodiments described herein, a piston and a plunger assembly cooperating with the piston. The dispensing device can further comprise a fastening unit or a loosening-unit for plugging a cap or an application nozzle to the dispensing device. Further, a mixer can be attachable to the dispensing device using a bayonet closure, a thread, or other fixations or moveable pre-mounted retaining-units or retaining-valves.

### BRIEF DESCRIPTION OF THE DRAWINGS

Exemplary embodiments of the present disclosure are described herein in conjunction with the following drawings, showing schematically:

FIG. 1 a dispensing device according to various embodiments;

FIG. 2A a cross sectional view of a round-shaped cartridge;

FIG. 2B a cross sectional view of an oval-shaped cartridge;

FIG. 2C a cross sectional view of a D-shaped cartridge;

FIG. 3A a grip plate arranged at the first end of the cartridge;

FIG. 3B a grip plate arranged at the second end of the cartridge;

FIG. 4A a lip and a plurality of grooves arranged on an outer surface of a cartridge;

FIG. 4B a cross sectional view of the cartridge of FIG. 4A;

FIG. 5A a locking rail arranged on an outer surface of a cartridge;

FIG. 5B a cross sectional view of the cartridge of FIG. 5A;

FIG. 6A a toothing arranged on an outer surface of a cartridge;

FIG. 6B a cross sectional view of the cartridge of FIG. 6A;

FIG. 7A a cross sectional view of a plurality of bulges arrange on an outer surface of a cartridge;

FIG. 7B a cross sectional view of a plurality of indentations arranged on an outer surface of a cartridge.

FIG. 8A a deformable grip plate according to an embodiment;

FIG. 8B a cross sectional view of the deformable grip plate of FIG. 8A;

FIG. 8C a side view of a dispensing device comprising the deformable grip plate of FIG. 8A;

FIG. 8D a cross sectional view of the dispensing device of FIG. 8C;

FIG. 9A a grip plate comprising a deformable cantilever according to an embodiment;

FIG. 9B a side view of the grip plate of FIG. 9A;

FIG. 9C a side view of a dispensing device comprising the grip plate of FIG. 9A;

FIG. 9D a cross sectional view of the dispensing device of FIG. 9C;

FIG. 10A a side view of a grip plate comprising a U-shaped channel according to an embodiment;

FIG. 10B a side view of a dispensing device comprising the grip plate of FIG. 10A;

FIG. 10C a perspective view of the dispensing device of FIG. 10B;

FIG. 10D a further perspective view of the dispensing device of FIG. 10B;

FIG. 11A a grip plate comprising division bars according to an embodiment;

FIG. 11B a cross sectional view of the grip plate of FIG. 11A;

FIG. 11C a top view of a dispensing device comprising the grip plate of FIG. 11A;

FIG. 11D a side view of the dispensing device of FIG. 11C;

FIG. 11E a cross sectional view of the dispensing device of FIG. 11C;

FIG. 12A a grip plate comprising an opening according to an embodiment;

FIG. 12B a perspective view of a dispensing device comprising the grip plate of FIG. 12A;

FIG. 12C a side view of the dispensing device of FIG. 12B;

FIG. 12D a cross sectional view of the dispensing device of FIG. 12C;

FIG. 13A a grip plate comprising an open tube according to an embodiment;

FIG. 13B a perspective view of a dispensing device comprising the grip plate of FIG. 13A;

FIG. 13C a further perspective view of a dispensing device comprising the grip plate of FIG. 13A;

FIG. 14A a perspective view of a dispensing device including a deformable grip plate arranged at one end of an open tube at a first position;

FIG. 14B a side view of the dispensing device of FIG. 14A;

FIG. 14C a cross sectional view of the dispensing device of FIG. 14B;

FIG. 15A a perspective view of the dispensing device of FIG. 14A at a second position;

FIG. 15B a side view of the dispensing device of FIG. 15A; and

FIG. 15C a cross sectional view of the dispensing device of FIG. 15B.

### DETAILED DESCRIPTION

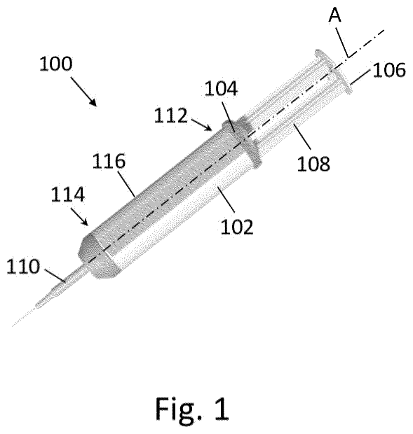

FIG. 1 shows a dispensing device 100 according to various embodiments. The dispensing devise 100 can be a syringe. The dispensing device 100 can include a cartridge 102, a grip plate 104, a plunger 106, a piston 108 and a needle 110.

The grip plate 104 can be at least initially arranged on an outer surface 116 of the cartridge 102 at a first end 112 of the cartridge 102. The grip plate 104 can be at least axially movable mounted at the cartridge 102 and axially fixable to the cartridge 102. The needle 110 can be arranged at a second end 114 of the cartridge 102. The cartridge 102 can be, for example, a cylindrical tube. The cartridge 102 can have a first opening at the first end 112 and a second opening at the second end 114.

The grip plate 104 can be axially movable between the first end 112 of the cartridge 102 and the second end 114 of the cartridge 102 in a direction along a longitudinal axis A. The longitudinal axis A can define a direction from the first end 112 of the cartridge 102 to the second end 114 of the cartridge 102. The grip plate 104 can be fixable at a position between the first end 112 of the cartridge 102 and the second end 114 of the cartridge 102 at the cartridge 102.

The first opening at the first end 112 of the cartridge 102 can be configured to receive the piston 108. The plunger 106 can be attached to a second end of the piston 108. A first end of the piston 108 can be oppositely arranged to the second end of the piston 108. The first end of the piston 108 can be configured to be inserted into the first end 112 of the cartridge 102. In an assembled state of the dispensing device 100, as shown in FIG. 1, the needle 110 can be attached to the second end 114 of the cartridge 102. Further, the first end of the piston 108 can be inserted into the first end 112 of the cartridge 102.

In FIG. 1, the cartridge 102 can be completely filled with a material, i.e. the cartridge 102 can contain the material from almost the first end 112 of the cartridge 102 to the second end 114 of the cartridge 102. In this context the term “almost” can mean that the material can not reach to the first opening at the first end 112 of the cartridge 102 since the first end of the piston 108 can be inserted into the first opening at the first end 112 of the cartridge 102. The first end of the piston 108 can contact the material inside the cartridge 102.

The material inside the cartridge 102 can be dispensed through the needle 110 by carrying out the following steps. A user, for example a dentist or a doctor, can grasp the dispensing device 100 by placing one finger, for example a thumb, on the plunger 106 and gripping the grip plate 104 with two other fingers, for example an index and a middle finger. The piston 108 can be pushed into the cartridge 102 in a direction along the longitudinal axis A by pressing the thumb on the plunger 106. The index finger and the middle finger can support the pressing of the plunger 106 with the thumb. A counterforce to a force that can be applied by pressing the thumb to the plunger 106 can be generated in that the index finger and the middle finger can be supported on the grip plate 104. By pressing the thumb on the plunger 106, the material inside the cartridge 102 can be squeezed out of the cartridge 102 through the second opening at the second end 114 of the cartridge 102, in particular through the needle 110 attached at the second end 114 of the cartridge 102.

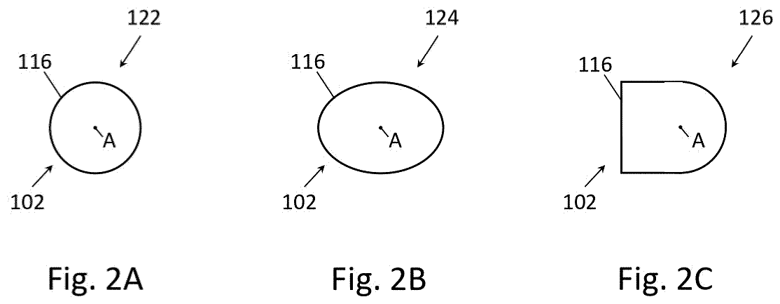

FIGS. 2A, 2B and 2C show cross sectional views of the cartridge 102 according to various embodiments. A cross section 122, 124, 126 of the cartridge 102 can be described by a section perpendicular to the longitudinal axis A of the cartridge 102.

FIG. 2A shows a round-shaped cross section 122 of a round-shaped cartridge 102. The round-shaped cross section 122 can describe a circle, particularly the round-shaped cross section 122 of the outer surface 116 of the round-shaped cartridge 102 can be a circle.

FIG. 2B shows an oval-shaped cross section 124 of an oval-shaped cartridge 102. The oval-shaped cross section 124 can describe an oval or can be of an elliptical shape, particularly the oval-shaped cross section 124 of the outer surface 116 of the oval-shaped cartridge 102 can be an oval.

FIG. 2C shows a D-shaped cross section 126 of a D-shaped cartridge 102. The D-shaped cross section 126 can describe a D-shape. The D-shaped cross section 126 can include a round-shaped section at one side of the D-shaped cross section 126 and a rectangular shape at the other side of the D-shaped cross section 126. Thus, the D-shaped cross section 126 of the outer surface 116 of the D-shaped cartridge 102 can look like an alphabetic character “D”.

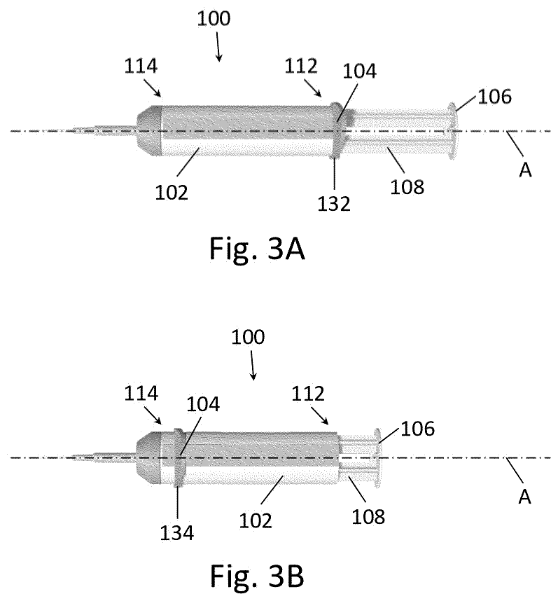

FIGS. 3A and 3B show a cartridge 102 with different positions of the grip plate 104 at the cartridge 102. FIG. 3A shows the grip plate 104 arranged at a first position 132 at the first end 112 of the cartridge 102. The grip plate 104 can be circumferentially closed and placed over the cartridge 102. Further, the piston 108 can be inserted into the cartridge 102 into the first opening of the cartridge 102. However, the piston 108 can only be inserted into the cartridge 102 by a small amount such that most of the piston 108 can protrude from the cartridge 102. This can be because the cartridge is completely filled with a material.

The grip plate 104 can be unlockable attached to the cartridge 102 to release a fixed distance between the first end 112 of the cartridge 102 and an axial position of the grip plate 104 or to release a fixed distance between the second end 114 of the cartridge 102 and the axial position of the grip plate 104. The grip plate 104 can be continuously moveable between the first end 112 of the cartridge 102 and the second end 114 of the cartridge 102 along the longitudinal axis A. In particular, the grip plate 104 can be continuously moveable from the first position 132 where the grip plate 104 is arranged at the first end 112 of the cartridge 102 (as shown in FIG. 3A) to a second position 134 where the grip plate 104 is arranged at the second end 114 of the cartridge 102 (as shown in FIG. 3B), and vice versa. The second position 134 can be arranged at any position between the first end 112 of the cartridge 102 and the second end 114 of the cartridge 102. Thus, the grip plate 104 can be movable back and forth in an axial direction along the longitudinal axis A of the cartridge 102. It can also be possible that the grip plate 104 is moveable in predefined steps between the first end 112 of the cartridge 102 and the second end 114 of the cartridge 102 along the longitudinal axis A.

FIG. 3B shows the grip plate 104 arranged at the second position 134 at the second end 114 of the cartridge 102. The grip plate 104 can be moved from the first position 132 to the second position 134 by releasing the grip plate 104 from the first position 132 (where the grip plate 104 can be fixed), moving the grip plate 104 from the first position 132 axially along the cartridge 102 in a direction defined by the longitudinal axis A to the second position 134, and fixing the grip plate 104 at the second position 134. The grip plate 104 can be fixable to the cartridge 102 at any position between the first end 112 of the cartridge 102 and the second end 114 of the cartridge 102, particularly at any position between the first position 132 of the cartridge 102 and the second position 134 of the cartridge 102.

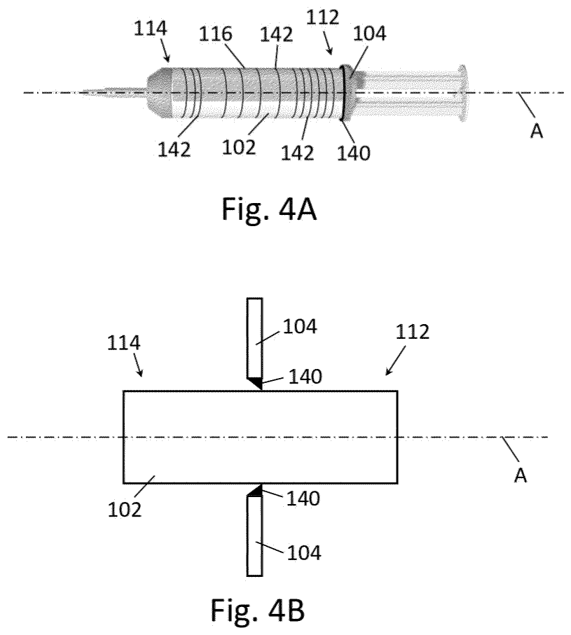

FIG. 4A shows a lip 140 and a plurality of grooves 142 arranged on an outer surface 116 of a cartridge 102. The lip 140 can be mounted on the grip plate 104. The cartridge 102 can include the plurality of grooves 142, wherein each groove 142 of the plurality of grooves 142 can be arranged in parallel to one other perpendicular to the longitudinal axis A on the outer surface 116 of the cartridge 102. Each groove 142 of the plurality of grooves 142 can be completely or partly circumferentially arranged at the cartridge 102. The plurality of grooves 142 can be arranged on the outer surface 116 of the cartridge 102 between the first end 112 of the cartridge 102 and the second end 114 of the cartridge 102. The plurality of grooves 142 can be equally spaced between the first end 112 of the cartridge 102 and the second end 114 of the cartridge 102. Additionally or alternatively, at least a portion of the plurality of grooves 142 can be arranged with different distances between two grooves 142 on the outer surface 116 of the cartridge 102.

Each groove 142 of the plurality of grooves 142 can be configured to receive the lip 140. Thus, each groove 142 of the plurality of grooves 142 can predefine a position or a step between the first end 112 of the cartridge 102 and the second end 114 of the cartridge 102. The grip plate 104 can be moveable between the first end 112 of the cartridge 102 and a second end 114 of the cartridge 102 along the longitudinal axis A of the cartridge 102 over the predefined steps defined by the plurality of grooves 142. The grip plate 104 can be fixable to the cartridge 102 at any predefined step between the first end 112 of the cartridge 102 and the second end 114 of the cartridge 102 by receiving the lip 140 through one groove 142 of the plurality of grooves 142.

FIG. 4B shows a cross sectional view of the cartridge 102 of FIG. 4A. The grip plate 104 can be fixable to the cartridge 102 using the lip 140. The lip 140 can be sloped on at least one side as shown in FIG. 4B. Alternatively, the lip 140 can be sloped on both sides of the lip 140. The grip plate 104 can be continuously moveable between the first end 112 of the cartridge 102 and the second end 114 of the cartridge 102 and the grip plate 104 can be fixable to the cartridge 102 at any position between the first end 112 of the cartridge 102 and the second end 114 of the cartridge 102 by clamping the lip 140 between the grip plate 104 and the cartridge 102.

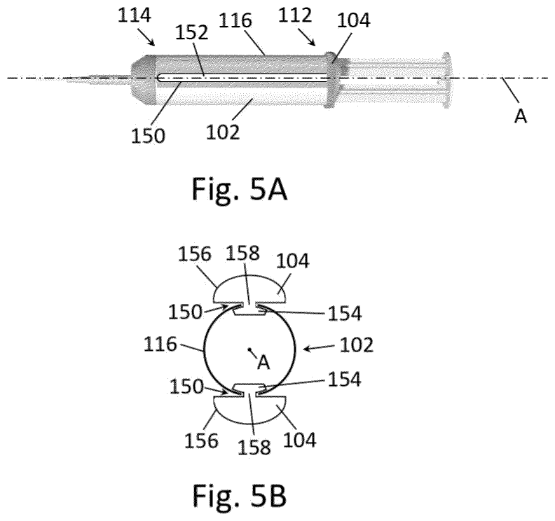

FIG. 5A shows a locking rail 150 arranged on the outer surface 116 of the cartridge 102. The locking rail 150 can include a longitudinal recess 152. The longitudinal recess 152 can extend in an axial direction along the longitudinal axis A of the cartridge 102 from the first end 112 of the cartridge 102 to the second end 114 of the cartridge 102. The grip plate 104 can be fixable to the cartridge 102 using the locking rail 150. The grip plate 104 can be moveable between the first end 112 of the cartridge 102 and the second end 114 of the cartridge 102 along the longitudinal axis A of the cartridge 102 by sliding the grip plate 104 along the locking rail 150. The grip plate 104 can be fixable to the cartridge 102 at any predefined step between the first end 112 of the cartridge 102 and the second end 114 of the cartridge 102 by clamping the grip plate 104 to the cartridge 102, for example, using a clamp or a latch (both not shown in FIG. 5A). The grip plate 104 can be moveable on the cartridge 102 by opening the clamp or the latch to unfix the grip plate 104 from the cartridge 102 before moving the grip plate 104 at least axially along the cartridge 102. After moving the grip plate 104 along the cartridge 102 to a desired position on the cartridge 102 between the first end 112 of the cartridge 102 and the second end 114 of the cartridge 102, the clamp or the latch can be closed to fix the grip plate 104 to the cartridge 102. The clamp or the latch can be configured to press the grip plate 104 to the cartridge 102 and thus, fix the grip plate 104 to the cartridge 102.

FIG. 5B shows a cross sectional view of the cartridge 102 of FIG. 5A. The cartridge 102 can include two oppositely arranged locking rails 150 arranged on the outer surface 116 of the cartridge 102. The grip plate 104 can include at least two oppositely arranged handles 156. The at least two oppositely arranged handles 156 can be square-shaped or round-shaped. It is understood, that the cartridge 102 can include only one locking rail 150 arranged on the outer surface 116 of the cartridge 102 and that the grip plate 104 can include only one handle 156. The only one handle 156 can be square-shaped or round-shaped.

The grip plate 104, or each of the grip plates 104, can further include a nose 154. The nose 154 can be embedded in the locking rail 150. The nose 154 can be attached to the grip plate 104 by a bar 158. The bar 158 can extend through the longitudinal recess 152 of the locking rail 150. The bar 158 can connect the nose 154 to the grip plate 104. Furthermore, the bar 158 can control a movement of the grip plate 104 in the locking rail 150 such that the grip plate 104 can only be moveable in the axial direction along the longitudinal axis A.

A width of the longitudinal recess 152 can be smaller than a width of the nose 154. Thus, a movement of the grip plate 104 perpendicular to longitudinal axis A can be avoided. The width of the longitudinal recess 152 can be a distance between two edges of the locking rail 150. The distance between two edges of the locking rail 150 can be measured perpendicular to the axial direction of the longitudinal axis A of the cartridge 102. The width of the nose 154 can also be measured perpendicular to the axial direction of the longitudinal axis A of the cartridge 102, wherein the distance of the nose 154 can be defined as a widest portion of the nose 154.

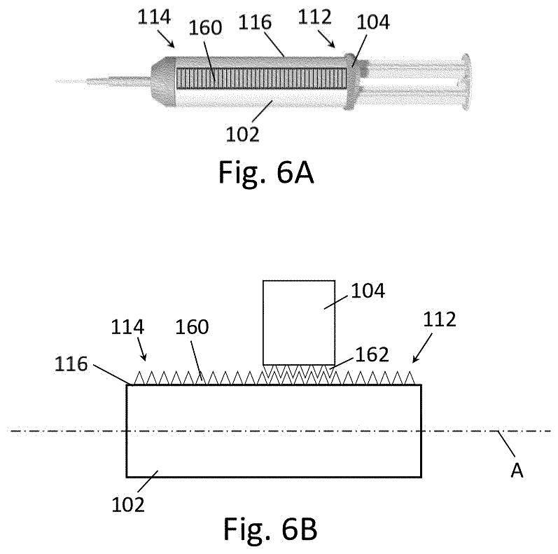

FIG. 6A shows a toothing 160 arranged on an outer surface 116 of the cartridge 102. The toothing 160 can include a plurality of teeth. The toothing 160 can extend in an axial direction along the longitudinal axis A of the cartridge 102 from the first end 112 of the cartridge 102 to the second end 114 of the cartridge 102. The grip plate 104 can be fixable to the cartridge 102 using the toothing 160. Therefore, the grip plate 104 can comprise at least one cam 162 (see FIG. 6B). The at least one cam 162 can include a plurality of teeth. The at least one cam 162 can be arranged on the grip plate 104 between the grip plate 104 and the toothing 160.

The grip plate 104 can be moveable between the first end 112 of the cartridge 102 and the second end 114 of the cartridge 102 along the longitudinal axis A of the cartridge 102 by sliding the grip plate 104 along the toothing 160. The at least one cam 162 and the toothing 160 can not be engaged when the grip plate 104 moves along the toothing 160.

FIG. 6B shows a cross sectional view of the cartridge 102 of FIG. 6A. The grip plate 104 can be fixable to the cartridge 102 at any predefined step, wherein the predefined step can be defined by the toothing 160 and the at least one cam 162, between the first end 112 of the cartridge 102 and the second end 114 of the cartridge 102 by clamping the grip plate 104 to the cartridge 102, for example, using a clamp or a latch (both not shown in FIG. 6B). The clamp or the latch can be configured to press the grip plate 104 to the cartridge 102 and thus, fix the grip plate to the cartridge 102. Particularly, the clamp or the latch can be configured to press the at least one cam 162 of the grip plate 104 to the toothing 160 of the cartridge 102. The clamp or the latch can be configured to fix the grip plate 104 to the cartridge 102 and unfix the grip plate 104 from the cartridge 102. The grip plate 104 can be moveable on the cartridge 102 by opening the clamp or the latch to unfix the grip plate 104 from the cartridge 102 before moving the grip plate 104 at least axially along the cartridge 102. After moving the grip plate 104 along the cartridge 102 to a desired position on the cartridge 102 between the first end 112 of the cartridge 102 and the second end 114 of the cartridge 102, the clamp or the latch can be closed to fix the grip plate 104 to the cartridge 102.

The toothing 160 and the at least one cam 162 can be equally toothed such that the teeth of the at least one cam 162 can mesh with at least a portion of the teeth of the toothing 160. The toothing 160 and the one or more cams 162 can mesh with one other if the grip plate 104 is fixed to the cartridge 102. FIG. 6B shows the toothing 160 and the at least one cam 162 in a fixed position.

In an embodiment, the at least one cam 162 can include at least one hook (not shown in the Figures), wherein the at least one hook can hook into the toothing 160. Thus, the grip plate 104 can be fixed to the cartridge 102 using the at least one hook.

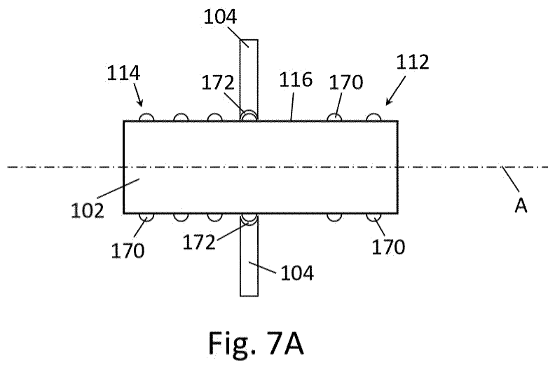

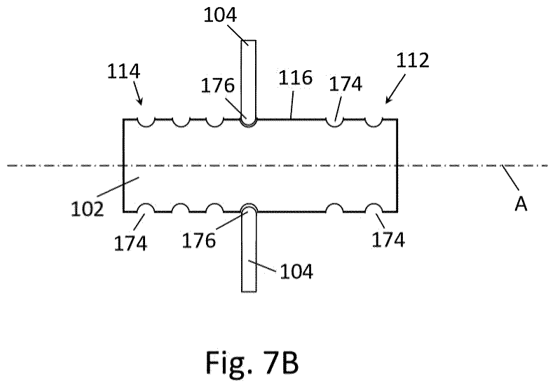

FIG. 7A shows a cross sectional view of a plurality of bulges 170 arranged on an outer surface 116 of a cartridge 102. The cartridge 102 can include the plurality of bulges 170, wherein each bulge 170 of the plurality of bulges 170 can be arranged in parallel to one other perpendicular to the longitudinal axis A on the outer surface 116 of the cartridge 102. Each bulge 170 of the plurality of bulges 170 can be completely or partly circumferentially arranged at the cartridge 102. The plurality of bulges 170 can be arranged on the outer surface 116 of the cartridge 102 between the first end 112 of the cartridge 102 and the second end 114 of the cartridge 102. The plurality of bulges 170 can be equally spaced between the first end 112 of the cartridge 102 and the second end 114 of the cartridge 102. Additionally or alternatively, at least a portion of the plurality of bulges 170 can be arranged with different distances between two bulges 170 on the outer surface 116 of the cartridge 102. Each bulge 170 of the plurality of bulges 170 can predefine a position or a step between the first end 112 of the cartridge 102 and the second end 114 of the cartridge 102.

The grip plate 104 can be fixable to the cartridge 102 using at least one bulge 170 of the plurality of bulges 170. Therefore, the grip plate 104 can comprise a recess 172. The recess 172 can be arranged on the grip plate 104 between the grip plate 104 and the outer surface 116 of the cartridge 102. The recess 172 can be configured to receive the at least one bulge 170.

The grip plate 104 can be moveable between the first end 112 of the cartridge 102 and the second end 114 of the cartridge 102 along the longitudinal axis A of the cartridge 102 by sliding the grip plate 104 along the outer surface 116 of the cartridge 102 over the predefined steps defined by the plurality of bulges 170. The recess 172 of the grip plate 104 can not receive one bulge 170 of the plurality of bulges 170 when the grip plate 104 moves along the outer surface 116 of the cartridge 102. The recess 172 of the grip plate 104 can receive one bulge 170 of the plurality of bulges 170 when the grip plate 104 moves over the bulge 170. Thus, the grip plate 104 can be fixed at the position of the bulge 170, when the recess 172 of the grip plate 104 can receive the bulge 170.

The grip plate 104 can be fixable to the cartridge 102 at any predefined step between the first end 112 of the cartridge 102 and the second end 114 of the cartridge 102. The recess 172 of the grip plate can receive one bulge 170 of the plurality of bulges 170 when the grip plate 104 is fixed to the one bulge 170 of the plurality of bulges 170.

FIG. 7B shows a cross sectional view of a plurality of indentations 174 arranged on an outer surface 116 of a cartridge 102. The cartridge 102 can include the plurality of indentations 174, wherein each indentation 174 of the plurality of indentations 174 can be arranged in parallel to one other perpendicular to the longitudinal axis A on the outer surface 116 of the cartridge 102. Each indentation 174 of the plurality of indentations 174 can be completely or partly circumferentially arranged at the cartridge 102. The plurality of indentations 174 can be arranged on the outer surface 116 of the cartridge 102 between the first end 112 of the cartridge 102 and the second end 114 of the cartridge 102. The plurality of indentations 174 can be equally spaced between the first end 112 of the cartridge 102 and the second end 114 of the cartridge 102. Additionally or alternatively, at least a portion of the plurality of indentations 174 can be arranged with different distances between two indentations 174 on the outer surface 116 of the cartridge 102. Each indentation 174 of the plurality of indentations 174 can predefine a position or a step between the first end 112 of the cartridge 102 and the second end 114 of the cartridge 102.

The grip plate 104 can be fixable to the cartridge 102 using at least one indentation 174 of the plurality of indentations 174. Therefore, the grip plate 104 can comprise a protrusion 176. The protrusion 176 can be arranged on the grip plate 104 between the grip plate 104 and the outer surface 116 of the cartridge 102. Each indentation 174 of the plurality of indentations 174 can be configured to receive the protrusion 176 of the grip plate 104.

The grip plate 104 can be moveable between the first end 112 of the cartridge 102 and a second end 114 of the cartridge 102 along the longitudinal axis A of the cartridge 102 by sliding the grip plate 104 along the outer surface 116 of the cartridge 102 over the predefined steps defined by the plurality of indentations 174. The protrusion 176 of the grip plate 104 can not be received by one indentation 174 of the plurality of indentations 174 when the grip plate 104 moves along the outer surface 116 of the cartridge 102. The protrusion 176 of the grip plate 104 can be received by one indentation 174 of the plurality of indentations 174 when the grip plate 104 moves over the indentation 174.

The grip plate 104 can be fixable to the cartridge 102 at any predefined step between the first end 112 of the cartridge 102 and the second end 114 of the cartridge 102. The protrusion 176 of the grip plate 104 can be received by one indentation 174 of the plurality of indentations 174 when the grip plate 104 is fixed to the one indentation 174 of the plurality of indentations 174.

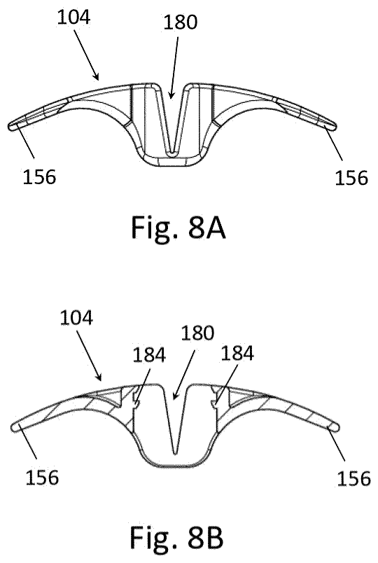

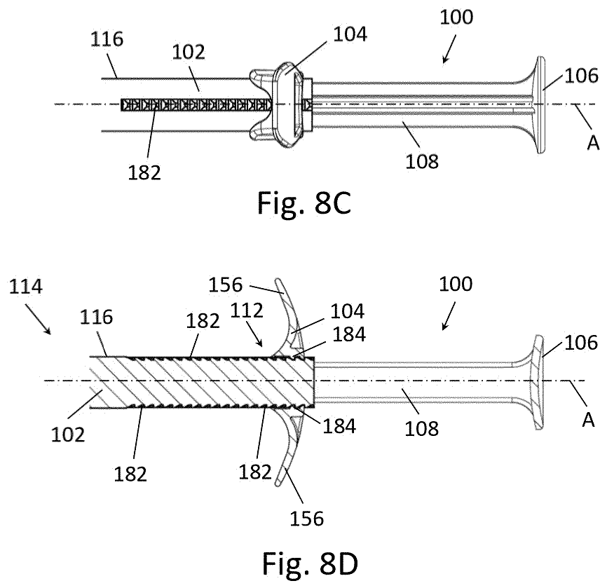

FIG. 8A shows a deformable grip plate 104 according to an embodiment. The grip plate 104 can include two oppositely arrange handles 156. The grip plate can further include a V-shaped gap 180 between the two oppositely arranged handles 156. The V-shaped gap 180 can be increase or decrease when the two oppositely arranged handles 156 can be pushed or pulled.

FIG. 8B shows a cross sectional view of the deformable grip plate 104 of FIG. 8A. The deformable grip plate 104 can further include at least one hook 184, in particular two oppositely arranged hooks 184. The at least one hook 184 can be arranged at an inner surface of the grip plate 104. The at least one hook 184 can extend from the inner surface of the grip plate 104 in a direction towards the V-shaped gap 180. The at least one hook 184 can move in a direction towards the V-shaped gap 180 or in a direction away from the V-shaped gap 180 when the two oppositely arranged handles 156 are pulled or pushed.

FIG. 8C shows a side view of a dispensing device 100 including the deformable grip plate 104 of FIG. 8A. The dispensing device 100 can further include a cartridge 102, a piston 108 and a plunger 106. The plunger 106 can be arranged at an end of the piston 108, wherein another end of the piston 108 can be inserted in the cartridge 102. The cartridge 102 can include a plurality of cavities 182 arranged in parallel to one another perpendicular to a longitudinal axis A on an outer surface 116 of the cartridge 102. The at least one hook 184 (shown in FIG. 8D) can engage at least one of the plurality of cavities 182 when the grip plate 104 is undeformed to fix the grip plate 104 to the cartridge 102.

FIG. 8D shows a cross sectional view of the dispensing device 100 of FIG. 8C. The grip plate 104 can be movable back and/or forth along an axial direction of the cartridge 102, in particular along the longitudinal axis A of the cartridge 102, when the grip plate 104 is in a deformed state and the grip plate 104 can be fixable to the cartridge 102 when the grip plate 104 is in an undeformed state. The two oppositely arranged hooks 184 can extend from the inner surface of the grip plate 104 in a direction perpendicular to the longitudinal axis A towards the longitudinal axis A of the cartridge 102. The inner surface of the grip plate 104 can face to the outer surface 116 of the cartridge 102, in particular the inner surface of the grip plate 104 can be in contact with the outer surface 116 of the cartridge 102 when the grip plate 104 is moving along the longitudinal axis A on the cartridge 102.

The grip plate 104 can be movable in a direction along the longitudinal axis A from a first position at a first end 112 of the cartridge 102 to a second position at a second end 114 of the cartridge 102 by pushing the two oppositely arranged handles 156 in the direction along the longitudinal axis A from the first position at the first end 112 of the cartridge 102 to the second position at the second end 114 of the cartridge 102. Thus, the two oppositely arranged hooks 184 can move away from the V-shaped gap 180 (see FIG. 8C) such that the two hooks 184 can not engage at least one of the plurality of cavities 182.

The grip plate 104 can be fixable at the second position on the cartridge 102 by stopping the pushing of the two oppositely arranged handles 156, for example by releasing a pressure on the two handles 156, and/or by pulling the two oppositely arranged handles 156 in a direction along the longitudinal axis A from the second position to the first position. Thus, the two oppositely arranged hooks 184 can move towards the V-shaped gap 180 such that the two hooks 184 can engage at least one of the plurality of cavities 182.

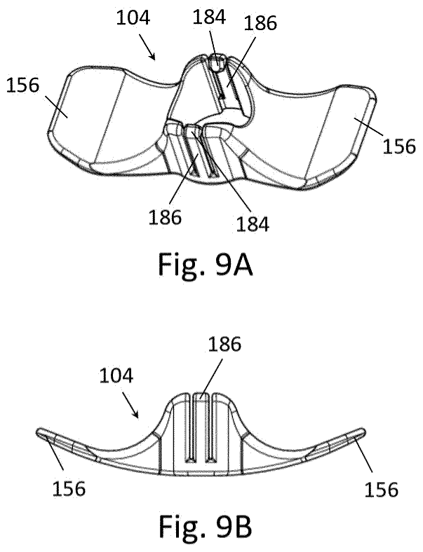

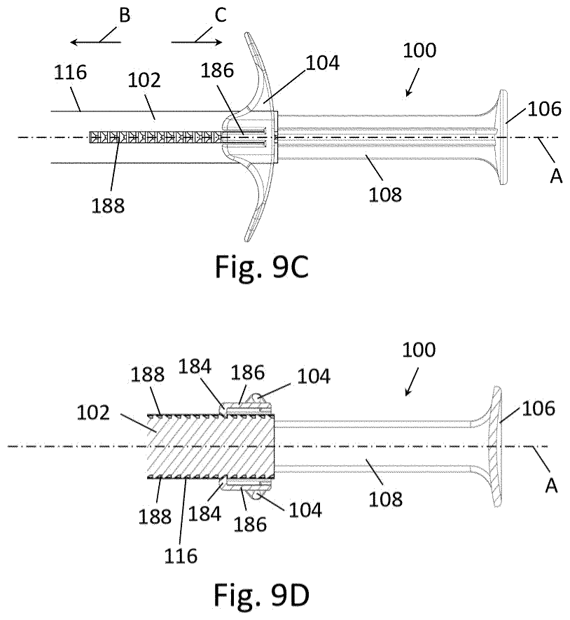

FIG. 9A shows a grip plate 104 including two deformable cantilever 186 according to an embodiment and FIG. 9B shows a side view of the grip plate 104 of FIG. 9A. Each of the two deformable cantilever 186 can be fixed to the grip plate 104 at one end of the respective deformable cantilever 186 and each of the deformable cantilever 186 can include a hook 184 at another end of the respective deformable cantilever 186. The respective hook 184 can be arranged at an inner surface of the grip plate 104. Each cantilever 186 of the two deformable cantilever 186 can be arrange between two oppositely arranged handles 156 of the grip plate 104.

FIG. 9C shows a side view of a dispensing device 100 including the grip plate 104 of FIG. 9A. The dispensing device 100 can further include a cartridge 102, a piston 108 and a plunger 106. The plunger 106 can be arranged at an end of the piston 108, wherein another end of the piston 108 can be inserted in the cartridge 102. The cartridge 102 can include a plurality of notches 188 arranged in parallel to one another perpendicular to a longitudinal axis A on an outer surface 116 of the cartridge 102. The at least one deformable cantilever 186, and therefore the grip plate 104, can be movable in a first direction B along an axial direction of the cartridge 102. The at least one deformable cantilever 186, and therefore the grip plate 104, can be fixable in a second direction C along the axial direction of the cartridge 102.

FIG. 9D shows a cross sectional view of the dispensing device 100 of FIG. 9C. The two oppositely arranged hooks 184 of the two oppositely arranged deformable cantilever 186 can be configured to slide over at least one of the plurality of notches 188 when the grip plate 104 is moving in the first direction B. The two oppositely arranged hooks 184 of the two oppositely arranged deformable cantilever 186 can be configured to engage at least one of the plurality of notches 188 when the grip plate 104 is fixed in the second direction C to the cartridge 102. The two oppositely arranged hooks 184 can extend from an inner surface of the grip plate 104 in a direction perpendicular to the longitudinal axis A of the cartridge 102 towards the longitudinal axis A of the cartridge 102. The inner surface of the grip plate 104 can face to an outer surface 116 of the cartridge 102, in particular the inner surface of the grip plate 104 can be in contact with the outer surface 116 of the cartridge 102 when the grip plate 104 is moving along the longitudinal axis A on the cartridge 102.

The grip plate 104 can be movable in a direction along the longitudinal axis A from a first position to a second position on the cartridge 102 by pushing the grip plate 104, in particular the two oppositely arranged handles 156 (see FIG. 9C) in the first direction B along the longitudinal axis A from the first position on the cartridge 102 to the second position on the cartridge 102. Thus, the two oppositely arranged hooks 184 can slide over the plurality of notches 188 arranged between the first position and the second position on the outer surface 116 of the cartridge 102 such that the two hooks 184 can not engage at least one of the plurality of notches 188.

The grip plate 104 can be fixable at the second position on the cartridge 102 by stopping the pushing of the two oppositely arranged handles 156, for example by releasing a pressure on the two handles 156, and/or by pulling the two oppositely arranged handles 156 in second direction C, i.e. a direction along the longitudinal axis A from the second position to the first position. Thus, the two oppositely arranged hooks 184 can engage at least one of the plurality of notches 188 at the second position.

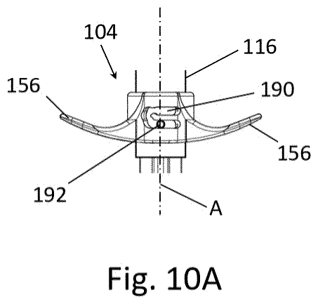

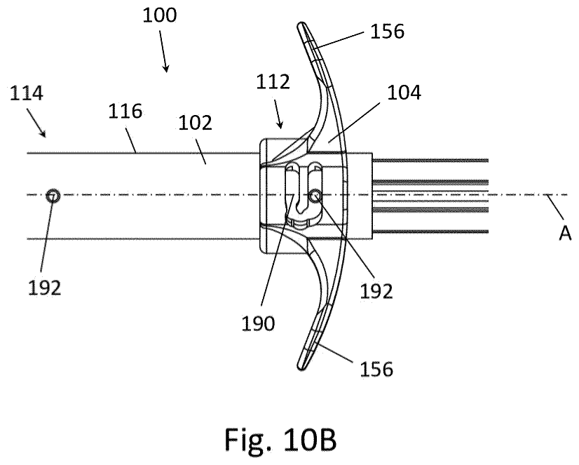

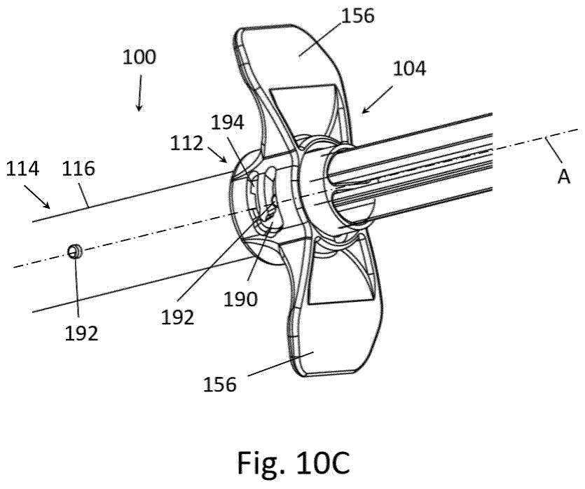

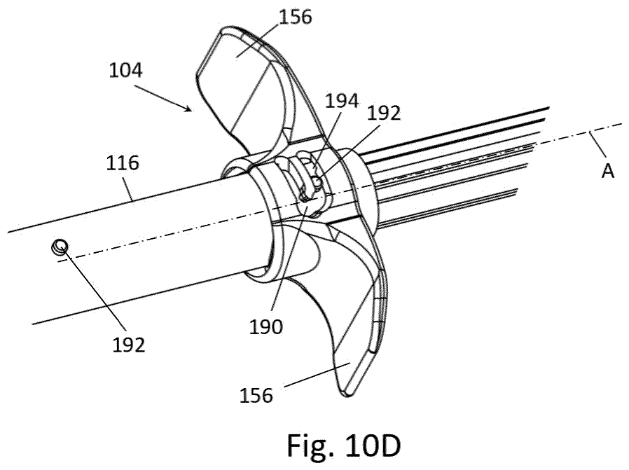

FIG. 10A shows a side view of a grip plate 100 including a U-shaped channel 190 according to an embodiment. The U-shaped channel 190 can be arranged between two oppositely arranged handles 156 of the grip plate 104. The grip plate 104 can further include at least one pin 192, wherein the at least one pin 192 can be arranged on an outer surface 116 of the cartridge 102. The U-shaped channel 190 can be configured to receive the at least one pin 192. The grip plate 104 can be rotatable about a longitudinal axis A of the cartridge 102. A rotation of the grip plate 104 can be controlled by the at least one pin 192 in the at least one U-shaped channel 190. It is understood that the grip plate 104 can include two oppositely arranged U-shaped channels 190, wherein each of the two oppositely arranged U-shaped channels 190 can be arranged between the two handles 156 of the grip plate 104.

FIG. 10B shows a side view of a dispensing device 100 including the grip plate 104 of FIG. 10A. The dispensing device 100 can include a cartridge 102 including a first pair of oppositely arranged pins 192 and a second pair of oppositely arranged pins 192. The first pair of oppositely arranged pins 192 can be located at a first end 112 of the cartridge 102 and the second pair of oppositely arranged pins 192 can be located at a second end 114 of the cartridge 102. The grip plate 104 can be moveable from the first pair of oppositely arranged pins 192 to the second pair of oppositely arranged pins 192 in a direction along a longitudinal axis A of the cartridge 102. The grip plate 104 can be fixable using the second pair of oppositely arranged pins 192. The grip plate 104 can be fixed at the first position as shown in FIG. 10B. Thus, the first pair of pins 192 is received by a pair of oppositely arranged U-shaped channels 190 of the grip plate 104. It is understood that also a first pin 192 located at the first end 112 of the cartridge 102 and a second pin 192 located at the second end 114 of the cartridge can be sufficient to fix the grip plate 104 to the cartridge 102 at the first position or the second position using the U-shaped channel 190 using one U-shaped channel 190 of the grip plate 104.

FIGS. 10C and 10D show perspective views of the dispensing device 100 of FIG. 10B. The grip plate 104 can further include two tubes 194. The tubes 194 can be configured to receive a pin 192, in particular the pin 192 can be slidable through the tubes 194 from outside the U-shaped channel 190 into the U-shaped channel 190, or vice versa. A first tube 194 of the two tubes 194 can be arranged at one end of the U-shaped channel 190. A second tube 194 (shown in FIG. 10D) of the two tubes 194 can be arranged at another end of the U-shaped channel 190.

To move the grip plate 104 from the first position defined by the first pair of pins 192 (or the first pin 192) arranged at the first end 112 of the cartridge 102 to the second position defined by the second pair of pins 192 (or the second pin 192) arranged at the second end 114 of the cartridge 102, the first pair of pins 192 (or the first pin 192) can be lead through the U-shaped channel 190 of the grip plate 104 from the one side of the U-shaped channel 190 to the other side of the U-shaped channel 190 by rotating the grip plate 104 about the longitudinal axis A until the first pair of pins 192 (or the first pin 192) can reach the other end of the U-shaped channel 190. The first pair of pins 192 (or the first pin 192) can be slide through the second tube 194 of the U-shaped channel 190. After sliding the first pin 192 through the U-shaped channel 190, there is no pin 192 located in the U-shaped channel 190 and the grip plate 104 is located between the firs pair of pins 192 (or the first pin 192) and the second pair of pins 192 (or the second pin 192).

The grip plate 104 can be moved on the cartridge 102 in a direction along the longitudinal axis A from the first position towards the second position. The second pair of pins 192 (or the second pin 192) located at the second end 114 of the cartridge 102 can be inserted into the first tube 194 and can be slide through the first tube 194 into the U-shaped channel 190. A fixation of the grip plate 104 using the U-shaped channel 190 and at least one pin 192 can act like a bayonet lock.

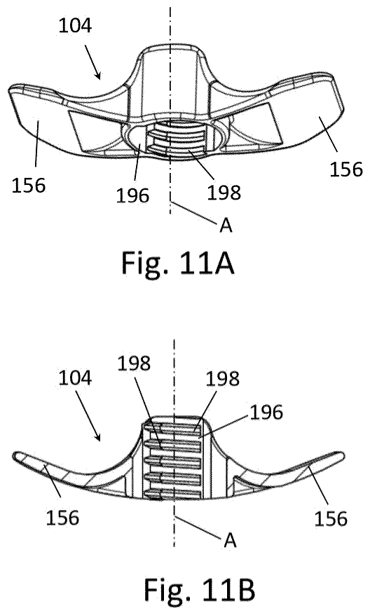

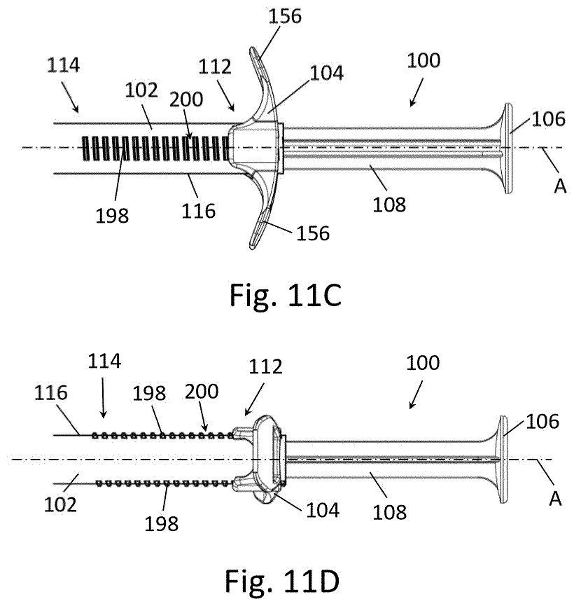

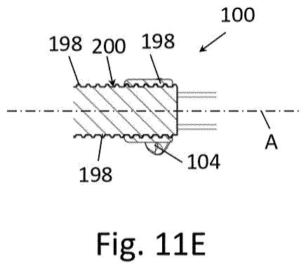

FIG. 11A shows a grip plate 104 including division bars 198 and FIG. 11B shows a cross sectional view of the grip plate 104 of FIG. 11A according to an embodiment. The grip plate 104 can include a first plurality of division bars 198 arranged in parallel to one another on an inner surface 196 of the grip plate 104 along a longitudinal axis A. The first plurality of division bars 198 can extend from the inner surface 196 of the grip plate 104 towards the longitudinal axis A. Each division bar 198 of the first plurality of division bars 198 can not be circumferentially closed and placed over the inner surface 196 of the grip plate 104. The first plurality of division bars 198 can be arranged between two oppositely arranged handles 156 of the grip plate 104.

FIG. 11C shows a top view, FIG. 11D shows a side view and FIG. 11E shows a cross sectional view of a dispensing device 100 including the grip plate 104 of FIG. 11A. The dispensing device 100 can further include a cartridge 102, a piston 108 and a plunger 106. The plunger 106 can be arranged at an end of the piston 108, wherein another end of the piston 108 can be inserted in the cartridge 102. The cartridge 102 can include a second plurality of division bars 198 arranged in parallel to one another on an outer surface 116 of the cartridge 102 along the longitudinal axis A of the cartridge 102.

The grip plate 104 can be rotatably arranged between a first rotational position and a second rotational position on the outer surface 116 of the cartridge 102. The first plurality of division bars 198 arranged on the inner surface 196 of the grip plate 104 can engage a plurality of indentations 200 formed between the second plurality of divisional bars 198 when the grip plate 104 is in the second rotational position. The first plurality of division bars 198 arranged on the inner surface 196 of the grip plate 104 can not engage the plurality of indentations 200 formed between the second plurality of divisional bars 198 when the grip plate 104 is in the first rotational position.

The grip plate 104 can be moveable along a direction to the longitudinal axis A of the cartridge 102 between a first end 112 of the cartridge 102 and a second end 114 of the cartridge 102 when the grip plate 104 is in the first rotational position. The grip plate 104 can be fixable at a position between the first end 112 of the cartridge 102 and the second end 114 of the cartridge 102 when the grip plate 104 is in the second rotational position.

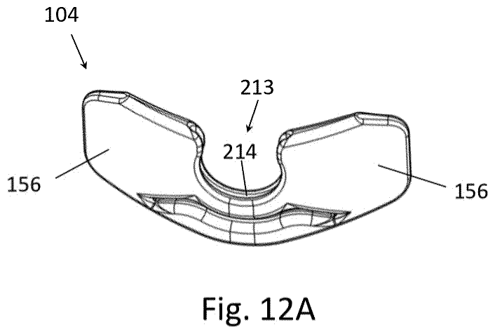

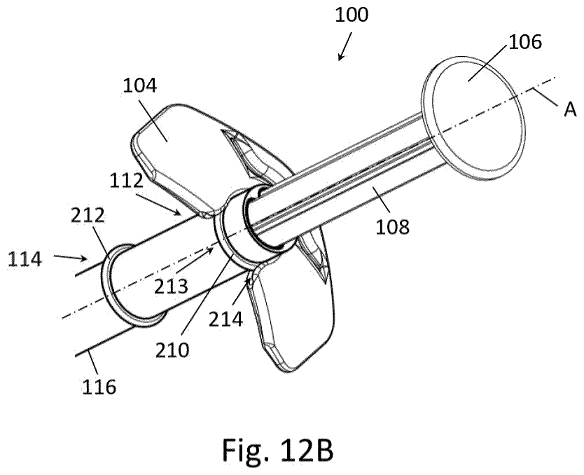

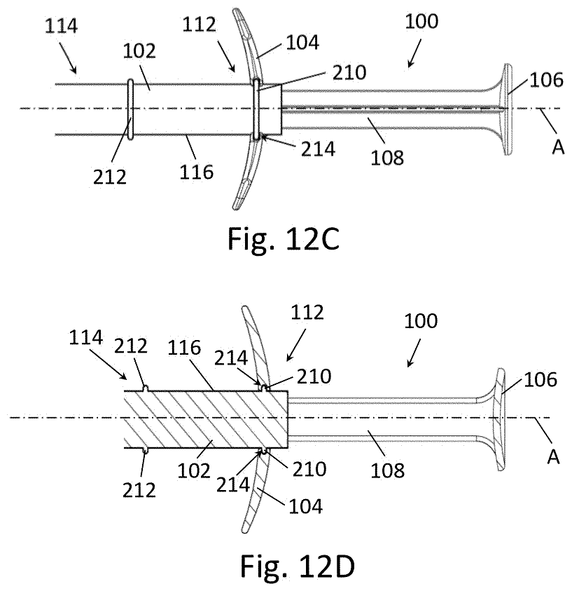

FIG. 12A shows a grip plate 104 including an opening 213 according to an embodiment. The opening 213 can be arranged between two oppositely arranged handles 156 of the grip plate 104. The opening 213 can be configured to receive a cartridge 102 as described herein. The grip plate 104 can further include a chamfer 214. The chamfer 214 can be arranged between the two oppositely arranged handles 156 of the grip plate 104. The chamfer 214 can start at one side of the opening 213 and end at another side of the opening 213. The chamfer 214 can be configured to receive at least a portion of a cartridge 102 as described herein.

FIG. 12B shows a perspective view, FIG. 12C shows a side view and FIG. 12D shows a cross sectional view of a dispensing device 100 including the grip plate 104 of FIG. 12A. The dispensing device 100 can further include a cartridge 102, a piston 108 and a plunger 106. The plunger 106 can be arranged at an end of the piston 108, wherein another end of the piston 108 can be inserted in the cartridge 102. The cartridge 102 can include two ridges 210, 212 arranged completely or partly circumferentially on an outer surface 116 of the cartridge 102. The two ridges 210, 212 can be arranged in parallel to one another on the outer surface 116 of the cartridge 102 along a longitudinal axis A of the cartridge 102. The chamfer 214 of the grip plate 104 can be configured to receive each ridge 210, 212 of the two ridges 210, 212 separately. The grip plate 104 can be configured to snap to one ridge 210, 212, either to a first ridge 210 arranged on the outer surface 116 at a first end 112 of the cartridge 102 or to a second ridge 212 arranged on the outer surface 116 at a second end 114 of the cartridge 102.

The grip plate 104 can enclose the cartridge 102 by more than 50% of a peripheral surface of the cartridge 102 when the grip plate 104 is snapped onto the first ridge 210 or onto the second ridge 212 on the outer surface 116 of the cartridge 102.

The grip plate 104 can be fixable at the first end 112 of the cartridge 102 by snapping the grip plate 104 onto the first ridge 210 such that the chamfer 214 can receive the first ridge 210. The grip plate 104 can be moveable between the first end 112 of the cartridge 102 and the second end 114 of the cartridge 102, or between the first ridge 210 and the second ridge 212, by removing the grip plate 104 from the first ridge 210 and snapping the grip plate 104 onto the second ridge 212. For example, by pressing the cartridge, in particular the second ridge 212 arranged on the outer surface 116 of the cartridge 102, through the opening 213 of the grip plate 104 into the chamfer 214 of the grip plate 104.

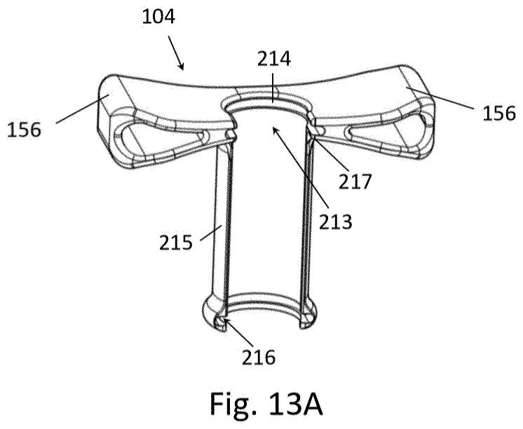

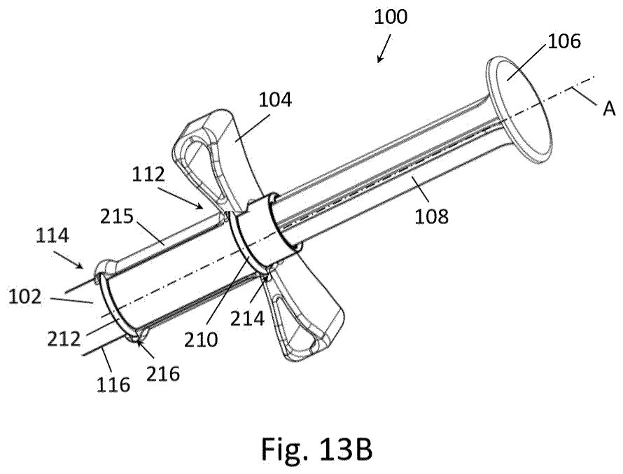

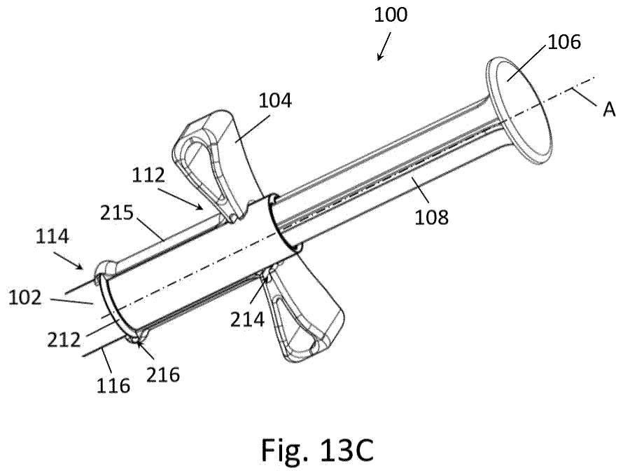

FIG. 13A shows a grip plate 104 including an open tube 215 according to an embodiment. The grip plate 104 of this embodiment can correspond to the grip plate of FIG. 12A. Thus, the grip plate can also include an opening 214, wherein the opening 213 can be arranged between two oppositely arranged handles 156 of the grip plate 104. The opening 213 can be configured to receive a cartridge 102 as described herein. The grip plate 104 can further include a chamfer 214. The chamfer 214 can be arranged between the two oppositely arranged handles 156 of the grip plate 104. The chamfer 214 can start at one side of the opening 213 and end at another side of the opening 213. The chamfer 214 can be configured to receive at least a portion of a cartridge 102 as described herein. Additionally, the grip plate 104 of FIG. 13A can include an open tube 215. The open tube 215 can extend from the grip plate 104 in a direction perpendicular to a surface including the two handles 156 of the grip plate 104. One end of the open tube 215 can attached to an edge 217 of the chamfer 214 on the grip plate 104, as shown in FIG. 13A. A second chamfer 216 can be arranged at another end of the open tube 215. The first chamfer 214 and the second chamfer 216 can be arranged in parallel to one another.

FIG. 13B shows a perspective view of a dispensing device 100 including the grip plate 104 of FIG. 13A. The dispensing device 100 can further include a cartridge 102, a piston 108 and a plunger 106. The plunger 106 can be arranged at an end of the piston 108, wherein another end of the piston 108 can be inserted in the cartridge 102. The cartridge 102 can include two ridges 210, 212 arranged completely or partly circumferentially on an outer surface 116 of the cartridge 102. The two ridges 210, 212 can be arranged in parallel to one another on the outer surface 116 of the cartridge 102 along a longitudinal axis A of the cartridge 102. The first ridge 210 can be arranged on an outer surface 116 of the cartridge 102 at a first end 112 of the cartridge 102. The second ridge 212 can be arranged on an outer surface 116 of the cartridge 102 at a second end 114 of the cartridge 102. The first chamfer 214 of the grip plate 104 can be configured to receive each ridge 210, 212 of the two ridges 210, 212 separately. The second chamfer 216 of the grip plate 104 can be configured to receive each ridge 210, 212 of the two ridges 210, 212 separately. The first chamfer 214 and the second chamfer 216 can be configured to receive the corresponding first ridge 210 and the corresponding second ridge 212.

The grip plate 104, in particular the open tube 215, can enclose the cartridge 102 by more than 50% of a peripheral surface of the cartridge 102 when the grip plate 104 or the open tube 215 is snapped onto the first ridge 210 and onto the second ridge 212 on the outer surface 116 of the cartridge 102.

The grip plate 104 can be fixable at the first end 112 of the cartridge 102 by snapping the first chamfer 214 of the grip plate 104 onto the first ridge 210 and snapping the second chamfer 216 of the grip plate 104 onto the second ridge 212 such that the first chamfer 214 can receive the first ridge 210 and the second chamfer 216 can receive the second ridge 212.

The grip plate 104 can be moveable between the first end 112 of the cartridge 102 and the second end 114 of the cartridge 102 by removing the first chamfer 214 of the grip plate 104 from the first ridge 210 and removing the second chamfer 216 of the grip plate 104 from the second ridge 212, and turning the grip plate 104 such that the first chamfer 214 of the grip plate 104 can receive the second ridge 212 and the second chamfer 216 of the grip plate 104 can receive the first ridge 210.

The grip plate 104 can be fixable at the second end 114 of the cartridge 102 by snapping the first chamfer 214 of the grip plate 104 onto the second ridge 212 and snapping the second chamfer 216 of the grip plate 104 onto the first ridge 210 such that the first chamfer 214 can receive the second ridge 212 and the second chamfer 216 can receive the first ridge 210.

FIG. 13C shows a further perspective view of a dispensing device 100 including the grip plate 104 of FIG. 13A. The dispensing device 100 can further include a cartridge 102, a piston 108 and a plunger 106. The plunger 106 can be arranged at an end of the piston 108, wherein another end of the piston 108 can be inserted in the cartridge 102. The cartridge 102 can include one ridge 212 arranged completely or partly circumferentially on an outer surface 116 of the cartridge 102. The ridge 212 can be arranged on an outer surface 116 of the cartridge 102 at a second end 114 of the cartridge 102. The first chamfer 214 of the grip plate 104 can be configured to receive the ridge 212. The second chamfer 216 of the grip plate 104 can be configured to receive the ridge 212.

The grip plate 104, in particular the open tube 215, can enclose the cartridge 102 by more than 50% of a peripheral surface of the cartridge 102 when the grip plate 104 or the open tube 215 is snapped onto the ridge 212 on the outer surface 116 of the cartridge 102.

The grip plate 104 can be fixable at the first end 112 of the cartridge 102 by snapping the second chamfer 216 of the grip plate 104 onto the ridge 212 such that the second chamfer 216 receives the ridge 212.

The grip plate 104 can be moveable between the first end 112 of the cartridge 102 and the second end 114 of the cartridge 102 by removing the second chamfer 216 of the grip plate 104 from the ridge 212, and turning the grip plate 104 such that the first chamfer 214 of the grip plate 104 can receive the ridge 212.

The grip plate 104 can be fixable at the second end 114 of the cartridge 102 by snapping the first chamfer 214 of the grip plate 104 onto the ridge 212 such that the first chamfer 214 can receive the ridge 212.

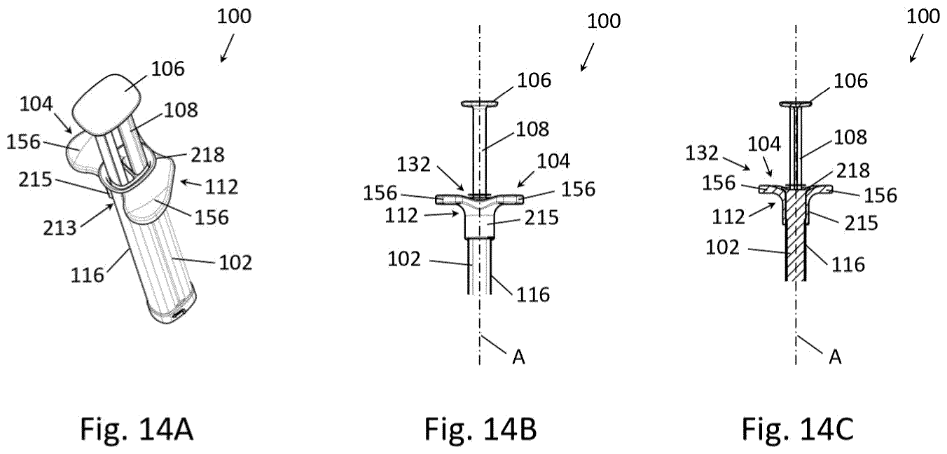

FIGS. 14A to 14C show a perspective view, a side view and a cross sectional view of a dispensing device 100 including a deformable grip plate 104 arranged at one end of an open tube 215 at a first position 132 according to an embodiment. The grip plate 104 and/or the open tube 215 can include an opening 213, wherein the opening 213 can be arranged between two oppositely arranged handles 156 of the grip plate 104. The opening 213 can be configured to receive a cartridge 102 as described herein. In particular, the open tube 215 can be configured to snap to an outer surface 116 of the cartridge 102. The open tube 215 can extend from the grip plate 104 at the one end of the open tube 215 in a direction perpendicular to a surface including the two handles 156 of the grip plate 104.

The dispensing device 100 can further include a cartridge 102, a piston 108 and a plunger 106. The plunger 106 can be arranged at an end of the piston 108, wherein another end of the piston 108 can be inserted in the cartridge 102. The cartridge 102 can include a rib 218 arranged completely or partly circumferentially at a first end 112 on the outer surface 116 of the cartridge 102. The grip plate 104, in particular the open tube 215, can enclose the cartridge 102 by more than 50% of a peripheral surface of the cartridge 102 when the grip plate 104 or the open tube 215 is snapped onto the outer surface 116 of the cartridge 102. The grip plate 104 and the open tube 215 can be in a deformed state during snapping onto the outer surface 116 of the cartridge 102. The grip plate 104 and the open tube 215 can remain on the cartridge 102 due to a friction force or a clamping force between the cartridge 102 and the open tube 215 when the open tube 215 is snapped onto the outer surface 116 of the cartridge 102. The grip plate 104 and the open tube 215 can be in an undeformed state when snapped onto the outer surface 116 of the cartridge 102.

The grip plate 104 can be fixable at a first position 132 at the first end 112 of the cartridge 102 by snapping the open tube 215 onto the outer surface 116 at the first end 112 of the cartridge 102 such that the one end of the open tube 215 contacts the rib 218.

The rib 218 can lock an axial movement of the grip plate 104 along the longitudinal axis A of the cartridge 102 during a dispensing of material by a holding force between the rib 218 and the one end of the open tube 215 that are in contact.

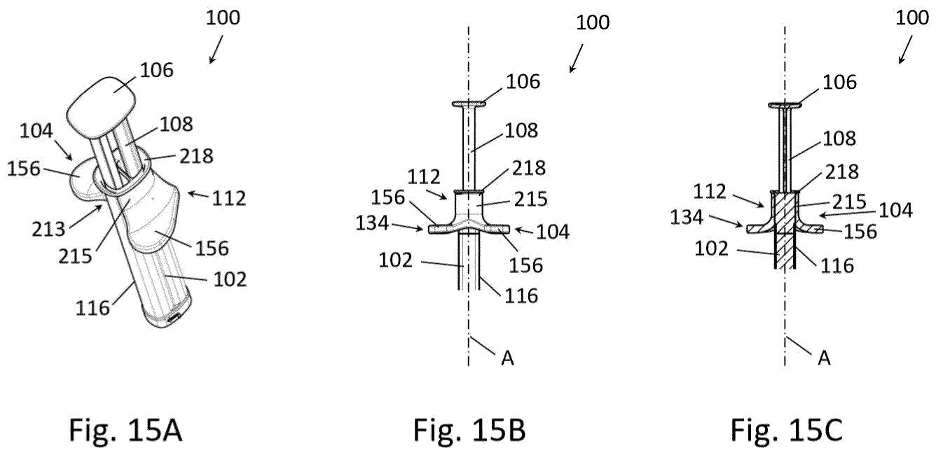

FIGS. 15A to 15C show a perspective view, a side view and a cross sectional view of the dispensing device 100 of FIGS. 14A to 14C at a second position 134 according to an embodiment. The grip plate 104 can be fixable at the second position 134 at the first end 112 of the cartridge 102 by snapping the open tube 215 onto the outer surface 116 at the first end 112 of the cartridge 102 such that another end of the open tube 215 oppositely arranged to the one end of the open tube 215 contacts the rib 218. The rib 218 can lock an axial movement of the grip plate 104 along the longitudinal axis A of the cartridge 102 during a dispensing of material by a holding force between the rib 218 and the other end of the open tube 215 that are in contact.

The grip plate 104 can be moveable between the first position 132 and the second position 134 by removing the open tube 215 from the cartridge 102, turning the open tube 215 by 180 degrees and snapping the open tube 215 onto the outer surface 116 at the first end 112 of the cartridge 102 again such that the other end of the open tube 215 that is oppositely arranged to the grip plate 104 contacts the rib 218.

The present disclosure can be directed at an optimized handling of a dispensing device 100, for example a syringe. An optimized ergonomic handling of the dispensing device 100 and a leverage of strength to easily dispense material out of the dispensing device 100 independent of a filling level of the dispensing device 100 can be reached by a movable and fixable grip plate 104 as described herein. A position of the grip plate 104 on an outer surface 116 of the cartridge 102 can be adapted by sliding the grip plate 104 along an axial direction on the outer surface 116 of the cartridge 102 for an optimal handling during different filling levels of the cartridge 102.

Further, the moveable and fixable grip plate 104 can allow to adapt the dispensing device 100 to needs of different users of the dispensing devise 100, for example, an easier use of the dispensing device 100 for females can be possible.

The idea of forming the grip plate 104 on a hand-held cartridge 102 moveable to adapt a distance between an end of the plunger 106 and the grip plate 104 can improve the usage of the dispensing device 100 depending on hand size and strength.

1. A cartridge for a dispensing device, comprising:
a grip plate,
the grip plate being at least axially moveably mounted at the cartridge and axially fixable to the cartridge.

2. The cartridge of claim 1, wherein a cross section of the cartridge perpendicular to an axial direction is round, oval, or D-shaped.

3. The cartridge of claim 1, wherein the cartridge comprises two containers arranged in parallel, the two containers having equal or unequal volumes or equal or unequal outer cross-sectional areas.

4. The cartridge of claim 1, wherein an outer shape of the cartridge is round or oval.

5. The cartridge of claim 1, wherein the grip plate is unlockable to release a fixed distance between one end of the cartridge and the grip plate.

6. The cartridge of claim 1, wherein the grip plate comprises at least two oppositely arranged handles.

7. The cartridge of claim 6, wherein the at least two oppositely arranged handles are square-shaped or round-shaped.

8. The cartridge of claim 1, wherein the grip plate is circumferentially closed and placed over the cartridge.

9. The cartridge of claim 1, wherein the grip plate is movable back or forth along an axial direction of the cartridge.

10. The cartridge of claim 1, wherein the grip plate is fixable to the cartridge using a lip.

11. The cartridge of claim 10, wherein the lip is mounted on the grip plate and the lip is arranged between the grip plate and the cartridge.

12. The cartridge of claim 10, wherein the lip is sloped on at least one side.

13. The cartridge of claim 10, wherein the grip plate is continuously moveable between a first end of the cartridge and a second end of the cartridge and the grip plate (104) is fixable to the cartridge at any position between the first end of the cartridge and the second end of the cartridge by clamping the lip between the grip plate and the cartridge.

14. The cartridge of claim 10, wherein the cartridge further comprises a plurality of grooves arranged in parallel to one other perpendicular to a longitudinal axis on an outer surface of the cartridge.

15. The cartridge of claim 14, wherein each groove of the plurality of grooves is configured to receive the lip.

16. The cartridge claim 14, wherein each groove of the plurality of grooves is completely or partly circumferentially arranged at the cartridge.

17. The cartridge of claim 14, wherein the grip plate is moveable between a first end of the cartridge and a second end of the cartridge along a longitudinal axis of the cartridge in predefined steps defined by the plurality of grooves and the grip plate is fixable to the cartridge at any predefined step between the first end of the cartridge and the second end of the cartridge by receiving the lip through one groove of the plurality of grooves.

18. The cartridge of claim 1. wherein the grip plate is fixable to the cartridge using a locking rail.

19. The cartridge of claim 18, wherein the locking rail comprises a longitudinal recess at an outer surface of the cartridge and the grip plate comprises a nose the nose is embedded in the locking rail.

20. The cartridge of claim 1, wherein the grip plate is fixable to the cartridge using a fixing device.

21. The cartridge of claim 20, wherein the fixing device comprises a toothing arranged at the cartridge and one or more cams arranged at the grip plate and the toothing and the one or more cams mesh with one another when the grip plate is fixed to the cartridge.

22. The cartridge of 20, wherein the fixing device comprises a latch to fix the grip plate to the cartridge and unfix the grip plate from the cartridge.

23. The cartridge claim 1, wherein the grip plate is fixable to the cartridge using at least one bulge or at least one indentation.

24. The cartridge of claim 23, wherein the at least one bulge or the at least one indentation is arranged completely or partly circumferentially at the cartridge.

25. The cartridge of claim 1, wherein the grip plate is deformable.

26. The cartridge of claim 26, wherein the grip plate comprises a V-shaped gap between two oppositely arranged handles.

27. The cartridge of claim 25, wherein the grip plate is movable back or forth along an axial direction of the cartridge when the grip plate is in a deformed state and the grip plate is fixable to the cartridge when the grip plate is in an undeformed state.

28. The cartridge of claim 25, wherein the cartridge comprises a plurality of cavities arranged in parallel to one another perpendicular to a longitudinal axis on an outer surface of the cartridge and the grip plate comprises at least one hook, and the at least one hook engages at least one of the plurality of cavities when the grip plate is undeformed to fix the grip plate to the cartridge.

29. The cartridge of claim 1, wherein the grip plate comprises at least one deformable cantilever.

30-69. (canceled)

70. A method of adapting a distance between an end of the cartridge in accordance with claim 1 and an actual position of a grip plate, the method comprising:
moving the grip plate at least axially along the cartridge, the grip plate being axially fixable to the cartridge,

71-88. (canceled)
